(19) United States
(12) Patent Application Publication
(10) Pub. No.: US 2026/0102430 A1
(43) Pub. Date: Apr. 16, 2026

(54) ORGANOID COMPOSITIONS HAVING IMMUNE CELLS

(71) Applicant: Children's Hospital Medical Center, Cincinnati, OH (US)

(72) Inventors: Michael A. Helmrath, Cincinnati, OH (US); Carine Bouffi, Danbury, CT (US)

(73) Assignee: Children's Hospital Medical Center, Cincinnati, OH (US)

(21) Appl. No.: 19/111,680

(22) PCT Filed: Sep. 13, 2023
(86) PCT No.: PCT/US2023/032650

§ 371 Date: Mar. 13, 2025

Related U.S. Application Data
(60) Provisional application No. 63/376,748, filed on Sep. 22, 2022.

(51) Int. Cl. A61K 35/17; G01N 33/50

(52) U.S. Cl. CPC A61K 35/17; G01N 33/5082; G01N 33/5091

(57) ABSTRACT

Disclosed herein are methods of generating intestinal organoids (IO) with immune cells (iIO) and methods of making an iIO with activated immune cells. IO compositions including immune cells, optionally including activated immune cells, (iIO) are also disclosed herein. Also disclosed are iIO models of disease states. Further disclosed are methods of treatment including transplanting iIO into an organism, as well as methods of screening a compound for activity using an iIO, for example an iIO model of a disease state.

### CROSS-REFERENCE TO RELATED APPLICATIONS

This application claims priority to and benefit of U.S. Provisional Patent Application No. 63/376,748, filed Sep. 22, 2022.

### STATEMENT REGARDING FEDERALLY SPONSORED R&D

This invention was made with government support under U01DK103117 awarded by the National Institutes of Health. The government has certain rights in the invention.

### FIELD OF THE INVENTION

Aspects of the present disclosure relate generally to intestinal organoid compositions having immune cells, methods of making and use thereof.

### BACKGROUND

The intestine represents the largest compartment of the immune system organized in the lamina propria, epithelium and lymphoid follicles defined by the gut-associated lymphoid tissue (GALT). Immune cell types and GALT are regionally expressed across the mucosal layers and along the gut tube. Immune-epithelial crosstalk is essential to maintain intestinal homeostasis and trigger a mechanism of host defense against pathogens or tolerance when exposed to dietary components or commensal bacteria. For instance, specialized epithelial cells called microfold (M) cells, expressed in the follicle-associated epithelium, play a central role by transporting luminal antigens in the lamina propria to activate immune cells. Immune-epithelial cross talk mediated by cytokines have been shown to play a key role in gastrointestinal (GI) tissue development, homeostasis as well as disease states.

### SUMMARY

Exemplary embodiments of the present disclosure are provided in the following numbered embodiments:
1. A method generating an intestinal organoid (IO) with immune cells (iIO) comprising transplanting an IO into an organism having immune cells for a period of time sufficient to form an IO having immune cells in one or more cell layers of the IO (iIO).
2. The method of embodiment 1, wherein the period of time is, is about, is at least, or is at least about, 6, 8, 10, 12, 14, 16, 18, or 20 weeks, or a range defined by any two of the preceding values, optionally, 6-20, 12-20 or 16-20 weeks.
3. The method of embodiment 1 or 2, wherein the period of time is, is about, is at least, or is at least about, 12 weeks or 16 weeks.
4. The method of any one of the preceding embodiments, wherein the iIO comprises CD4+ T cells and/or CD20+ B cells localized to lamina propria and epithelium.
5. The method of any one of the preceding embodiments, wherein the iIO comprises aggregates of T and B cells.
6. The method of any one of the preceding embodiments, wherein the iIO comprises CD4+ T cells and CD8+ T cells in a T-cell zone.
7. The method of any one of the preceding embodiments, wherein the iIO comprises plasma cells and neutrophils.
8. The method of any one of the preceding embodiments, wherein the iIO expresses gut-associated lymphoid tissue (GALT)-associated chemokines.
9. The method of embodiment 8, wherein the GALT-associated chemokines are CCL19, CCL21, and/or CXCL13.
10. The method of any one of the preceding embodiments, wherein the iIO comprises GALT-associated B cells.
11. The method of embodiment 10, wherein the B cells are aggregated in a lymphoid-like structure.
12. The method of any one of the preceding embodiments, wherein the iIO comprises CD45+ cells.
13. The method of embodiment 12, wherein the CD45+ cells are localized to a mucosal layer, lamina propria, and/or epithelium of the iIO.
14. The method of embodiment 12 or 13, wherein the CD45+ cells form cellular aggregates.
15. The method of any one of the preceding embodiments, wherein the iIO comprises CD3+ B cells and CD20+ T cells.
16. The method of any one of the preceding embodiments, wherein the iIO comprises CD4+ T cells, innate lymphoid cells, mucosal-associated invariant T (MAIT)-like cells, CD8+ T cells, lymphoid tissue inducer-like cells, B cells, natural killer cells, dendritic cells, intraepithelial lymphocytes, macrophages, natural killer T cells, and/or neutrophils.
17. The method of any one of the preceding embodiments, wherein the iIO comprises enterocytes, goblet cells, Paneth cells, and enteroendocrine cells.
18. The method of any one of the preceding embodiments, wherein the iIO comprises Villin+, Mucin2+, Lysozyme+, and/or Chromogranin A+ cells.
19. The method of any one of the preceding embodiments, wherein upon exposure of the lumen of the iIO to a lysate of Escherichia coli, microfold cells (M cells) of the iIO express glycoprotein 2 (GP2) at the cell surface.
20. The method of any one of the preceding embodiments, wherein upon exposure of the lumen of the iIO to a lysate of Escherichia coli, plasma cells of the iIO produce IgA antibodies.
21. The method of any one of the preceding embodiments, wherein upon exposure of the lumen of the iIO to a lysate of Escherichia coli, M cells and B cells of the iIO are co-localized.
22. The method of any one of the preceding embodiments, wherein the organism is not human.
23. The method of any one of the preceding embodiments, wherein the IO is transplanted into a kidney capsule of the organism.
24. The method of any one of the preceding embodiments, wherein the organism does not have Peyer's patches or lymphoid follicles in an intestine.
25. The method of any one of the preceding embodiments, wherein the organism is a mouse.
26. The method of embodiment 25, wherein the mouse is a lymphopenic mouse
27. The method of embodiment 26, wherein the mouse is a NOD/SCID/Il2rg−/− mouse with transgenic expression of human SCF, GM-CSF, and IL-3 (NSGS mouse).
28. The method of any one of the preceding embodiments, wherein prior to transplanting of the IO, the mouse is injected with cord blood cells, optionally wherein the cord blood cells are human cord blood cells.
29. The method of embodiment 28, wherein the mouse is treated with a pharmaceutical compound prior to the injection of cord blood cells to optimize engraftment of cord blood cells, optionally wherein the pharmaceutical compound depletes mouse bone marrow cells.
30. The method of any one of the preceding embodiments, wherein the IO is made by a method comprising:
a) exposing definitive endoderm (DE) cells to an FGF activator and a Wnt pathway activator for a period of time to differentiate into a hindgut spheroid, optionally a mid-hindgut spheroid;
b) embedding the hindgut spheroid into a basement membrane matrix; and
c) exposing the embedded hindgut spheroid to EGF for a period of time to differentiate into an IO, optionally wherein the IO is a human IO (HIO).
31. The method of any one of the preceding embodiments, wherein the method further comprises, prior to implanting the IO, making the IO by a method comprising:
a) exposing definitive endoderm (DE) cells to an FGF activator and a Wnt pathway activator for a period of time to differentiate into a hindgut spheroid, optionally a mid-hindgut spheroid;
b) embedding the hindgut spheroid into a basement membrane matrix; and
c) exposing the embedded hindgut spheroid to EGF for a period of time to differentiate into an IO, optionally wherein the IO is an HIO.
32. The method of embodiment 30 or 31, wherein the DE cells are derived from pluripotent stem cells (PSCs), optionally wherein the PSCs are embryonic stem cells (ESCs) or induced pluripotent stem cells (iPSCs), optionally wherein the PSCs are human PSCs.
33. The method of any one of embodiments 30-32, wherein the basement membrane matrix environment is Matrigel.
34. The method of any one of embodiments 30-33, wherein the FGF activator is FGF4, optionally wherein the concentration is, is about, is at least, or is at least about 50 ng/ml, 100 ng/ml, 150 ng/ml, 200 ng/ml, 250 ng/ml, 300 ng/ml, 350 ng/ml, 400 ng/ml, 450 ng/ml, 500 ng/ml, 550 ng/ml, 600 ng/ml, 650 ng/ml, 700 ng/ml, or 750 ng/ml, or a range defined by any two of the preceding values, optionally 50-750 ng/ml, 50-100 ng/ml, or 50-500 ng/ml, or optionally at a concentration of 500 ng/ml.
35. The any one of embodiments 30-34, wherein the Wnt pathway activator is CHIRON 99021, optionally wherein the concentration is, is about, is at least, or is at least about 0.5 μM, 1 μM, 1.5 μM, 2 μM, 2.5 μM, 3 μM, 3.5 μM, 4 μM, 4.5 μM, 5 μM, 5.5 μM, or 6 μM, or a range defined by any two of the preceding values, optionally 0.5-6 μM, 0.5-3 μM, 3-6 μM, 2-4 μM, or optionally at a concentration of 3 μM.
36. The method of any one of embodiments 30-35, wherein the concentration of EGF is, is about, is at least, or is at least about 25 ng/ml, 50 ng/ml, 75 ng/ml, 100 ng/ml, 125 ng/ml, 150 ng/ml, 175 ng/ml, or 200 ng/ml, or a range defined by any two of the preceding values, optionally 25-100 ng/ml, 50 -150 ng/ml, 100 ng/ml, or optionally is at a concentration of 100 ng/ml.
37. The method of any of the preceding embodiments, wherein the IO is matured in vitro for a period of time prior to transplantation, optionally wherein the period of time is, is about, is at least, or is at least about, 7, 10, 14, 16, 21, 25, or 28 days, or a range defined by any two of the preceding values, optionally 7-28, 14-28, or 21-28 days.
38. A method of making an iIO with activated immune cells comprising administering an immune stimulating material to a lumen of the iIO.
39. The method of embodiment 38, wherein the iIO is made by the method of any one of embodiments 1-37.
40. The method of embodiment 38 or 39, wherein the immune stimulating material is an allergen, and/or microbial lysate, optionally wherein the microbial lysate comprises Escherichia coli.
41. The method of any one of embodiments 38-40, wherein the iIO with activated immune cells comprises GP2+ microfold (M) cells after a period of time post-administration of the microbial lysate.
42. The method of embodiment 41, wherein the period of time post-administration is, is about, is at least, or is at least about, 24, 36, 48, 60, or 72 hours.
43. The method of any one of embodiments 38-42, wherein mucus of the iIO with activated immune cells comprises secreted IgA antibodies.
44. The method of any one of embodiments 38-43, wherein M cells and B cells colocalize within the iIO with activated immune cells.
45. An iIO with activated immune cells made by the method of any one of embodiments 38-44.
46. The method of any one of the preceding embodiments, wherein the IO is a human IO (HIO).
47. The method of any one of the preceding embodiments, wherein the immune cells of the organism comprise human immune cells, and/or the organism has a humanized immune system.
48. The method of any one of the preceding embodiments, wherein the IO is an HIO, wherein the organism has human immune cells and/or a humanized immune system, and wherein the HIO comprises human immune cells and/or humanized immune cells in one or more layers of the HIO (hiHIO).
49. An iIO made by the method of any one of the preceding embodiments.
50. An IO comprising immune cells in one or more cell layers of the IO (iIO).
51. The iIO of embodiment 50, wherein the iIO comprises CD4+ T cells and/or CD20+ B cells localized to lamina propria and epithelium.
52. The iIO of any one of embodiments 50-51, wherein the iIO comprises aggregates of T and B cells.
53. The iIO of any one of embodiments 50-52, wherein the iIO comprises CD4+ T cells and CD8+ T cells in a T-cell zone.
54. The iIO of any one of embodiments 50-53, wherein the iIO comprises plasma cells and neutrophils.
55. The iIO of any one of embodiments 50-54, wherein the iIO expresses gut-associated lymphoid tissue (GALT)-associated chemokines.
56. The iIO of embodiment 55, wherein the GALT-associated chemokines are CCL19, CCL21, and/or CXCL13.
57. The iIO of any one of embodiments 50-56, wherein the iIO comprises GALT-associated B cells.
58. The iIO of embodiment 57, wherein the B cells are aggregated in a lymphoid-like structure.
59. The iIO of any one of embodiments 50-58, wherein the iIO comprises CD45+ cells.
60. The iIO of embodiment 59, wherein the CD45+ cells are localized to a mucosal layer, lamina propria, and/or epithelium of the iIO.
61. The iIO of embodiment 59 or 60, wherein the CD45+ cells form cellular aggregates.
62. The iIO of any one of embodiments 50-61, wherein the iIO comprises CD3+ B cells and CD20+ T cells.
63. The iIO of any one of embodiments 50-62, wherein the iIO comprises CD4+ T cells, innate lymphoid cells, mucosal-associated invariant T (MAIT)-like cells, CD8+ T cells, lymphoid tissue inducer-like cells, B cells, natural killer cells, dendritic cells, intraepithelial lymphocytes, macrophages, natural killer T cells, and/or neutrophils.
64. The iIO of any one of embodiments 50-63, wherein the iIO comprises enterocytes, goblet cells, Paneth cells, and enteroendocrine cells.
65. The iIO of any one of embodiments 50-64, wherein the iIO comprises Villin+, Mucin2+, Lysozyme+, and/or Chromogranin A+ cells.
66. The iIO of any one of embodiments 50-65, wherein upon exposure of the lumen of the iIO to a lysate of Escherichia coli, microfold cells (M cells) of the iIO express GP2 at the cell surface.
67. The iIO of any one of embodiments 50-66, wherein upon exposure of the lumen of the iIO to a lysate of Escherichia coli, plasma cells of the iIO produce IgA antibodies.
68. The iIO of any one of embodiments 50-67, wherein upon exposure of the lumen of the iIO to a lysate of Escherichia coli, M cells and B cells of the iIO are co-localized.
69. The iIO of any one of embodiments 50-68, comprising activated immune cells.
70. The iIO of embodiment 69, wherein the iIO comprises GP2+ microfold (M) cells.
71. The iIO of any one of embodiments 69-70, wherein mucus of the iIO comprises secreted IgA antibodies.
72. The iIO of any one of embodiments 69-71, wherein M cells and B cells colocalize within the iIO.
73. The iIO of any one of embodiments 49-72, wherein the IO is an HIO.
74. The iIO of any one of embodiments 49-73, wherein the immune cells comprise human and/or humanized immune cells.
75. The iIO of any one of embodiments 49-74, wherein the IO is an HIO, and wherein the HIO comprises human immune cells and/or humanized immune cells in one or more layers of the HIO (hiHIO).
76. The method or iIO of any one of the preceding embodiments, wherein the iIO resembles at least one GI disease state.
77. The method or iIO of embodiment 76, wherein the GI disease state is an allergy and/or infectious disease.
78. The method or iIO of embodiment 76 or 77, wherein the GI disease state is induced by a genetic modification of one or more cell types of the iIO.
79. The method or iIO of any one of embodiments 76-78, wherein the GI disease state is induced by exposure to a compound, an allergen, and/or a pathogen.
80. The method or iIO of any one of the preceding embodiments, wherein the iIO is utilized to develop a mucosal vaccine.
81. A method of treatment comprising transplanting the iIO of any one of embodiments 49-75 into an organism, optionally wherein the organism is suffering from a GI disease state.
82. A method of screening a compound for activity comprising contacting the iIO of any one of embodiments 49-79 with the compound and measuring a response of the iIO to the compound.

### BRIEF DESCRIPTION OF THE DRAWINGS

In addition to the features described herein, additional features and variations will be readily apparent from the following descriptions of the drawings and exemplary embodiments. It is to be understood that these drawings depict embodiments and are not intended to be limiting in scope.

FIG. 1A depicts an embodiment of experimental workflow. Human intestinal organoids (HIOs) generated in vitro were then transplanted in humanized or control mice. At 12, 16 and 20 weeks post transplantation, HIOs and tissues of interest were collected for analysis.

FIGS. 1B-D depicts an embodiment of flow cytometry analysis of peripheral blood from humanized mice. Contour plots represent the gating strategy of human immune cell lineages in humanized mice post HIO transplantation (FIG. 1B). Graph represents percentage of total hCD45+ cells (FIG. 1C) or, as indicated, immune cell subsets (FIG. 1D) in peripheral blood of humanized mice at 12, 16 and 20 weeks post HIO transplantation.

FIG. 1E depicts an embodiment of a graph representing the relationship between length of HIO and percentage of blood hCD45 at different time points. Pearson correlation coefficient r=0.1881 with p value=0.4405.

FIG. 1F depicts an embodiment of humanized mouse small intestine at indicated time, were stained with antihuman CD45 by immunohistochemistry(IHC) and counterstained with hematoxylin. Scale bar represents 50 μm.

FIG. 2A depicts an embodiment of transplanted HIO at 12,16 and 20 weeks with mouse kidney seen underneath from control or humanized mice.

FIG. 2B depicts an embodiment of a graph representing the length of HIOs from control (grey triangle) or humanized (red triangle) group at 12 weeks (n=6 control and 7 humanized mice), 16 weeks (n=9 control and 9 humanized mice) post-transplantation and 20 weeks (n=4 control and 3 humanized mice). Mean+/− standard deviation (SD). Multiple Mann-Whitney tests; p=0.1666 for 12 weeks, p=0.7120 for 16 weeks and p=0.714 for 20 weeks; non-significant (n.s). Graph representative of at least 3 independent experiments.

FIG. 2C depicts an embodiment of a formalin-fixed paraffin-embedded (FFPE) sections of transplanted HIO at 12, 16 and 20 weeks stained by IHC with anti-human CD45 antibody. Scale bar represents 100 μm.

FIG. 2D depicts an embodiment of human fetal intestine at 14.7 and 20.7 post conception week (PCW) stained, by immunofluorescence, with anti-human CDH1 (E-cadherin) (blue), anti-human CD45 (green) antibodies and DAPI (white). Scale bar represents 100 μm.

FIG. 2E depicts an embodiment of a human adult jejunum stained by IHC with anti-human CD45 antibody. Scale bar represents 100 μm.

FIG. 3A depicts an embodiment of a heatmap illustrating the level of expression of each marker (x axis) for each cluster corresponding to their identified cell type (y axis).

FIG. 3B depicts an embodiment of a visualization of high-dimensional data with UMAP (Uniform Manifold Approximation and Projection) overlaid with identified cell types. Heatmap (FIG. 3A) and UMAP (FIG. 3B) were generated with combined CyTOF data set from transplanted HIO and humanized mouse small intestine (SI) at 12, 16 and 20 weeks post transplantation.

FIG. 3C depicts an embodiment of a stacked bar graph representing the percentage of each cell types per tissue and time point.

FIGS. 4A-B depicts an embodiment of a HIO at 12, 16 and 20 weeks post-transplantation stained with anti-human CD3 (FIG. 4A) or CD20 (FIG. 4B). Scale bar represents 100 μm.

FIG. 5A depicts an embodiment of prevalence of immune developmental features observed in HIO.

FIGS. 5B-F depict an embodiment of images illustrating features observed in HIO that resemble immune cell development described in fetal gut. FIGS. 5B-D depict an embodiment of HIO sections stained with antihuman CD3 (top image), anti-human CD4/CD8 (middle image) and anti-human CD20 (bottom image). FIG. 5E depicts an embodiment of H&E staining indicating the presence of neutrophils (black arrowheads) observed in late developing HIOs only. FIG. 5F depicts an embodiment of H&E staining highlight the presence of plasma cells (black arrowheads) then confirmed with anti-MUMI immunohistochemical staining (bottom image). Scale bar represents 50 μm.

FIGS. 6A-C depicts an embodiment of 16-week HIO sections at 72 hours post-injection with saline (left) or E. coli lysate (right) stained with antihuman CD45 (FIG. 6A), anti-hGP2 (FIG. 6B) and anti-hMUMI (FIG. 6C). Arrows indicate M cells positive for hGP2. Scale bar represents 100 μm, except FIG. 6B (bottom image) hGP2 image scale bar represents 50 μm.

FIG. 6D depicts an embodiment of a graph representing level of GP2 gene expression evaluated by qPCR in 16-week HIO at 72 hours post injection with saline or E. coli lysate. Saline group n=3 transplanted HIOs and E. coli lysate group n=6 transplanted HIOs. GP2 expression is normalized to GAPDH gene. Mean+/− standard deviation (SD). Mann-Whitney tests; p=0.1667; non-significant (n.s.).

FIG. 6E depicts an embodiment of a level of human IgA measured by ELISA in mucus from 16-week HIO 72 hours after being injected with saline (n=2) or E. coli lysate (n=4). Mean+/− standard deviation (SD). Mann-Whitney tests; p=0.5333; non-significant (n.s.).

FIG. 6F depicts an embodiment of immunofluoresence staining with anti-CDH1 (white), anti-hGP2 (red) and anti-hCD20 (blue) in 16-week HIOs injected with E. coli lysate for 72 hours. Scale bar represents 50 μm.

FIGS. 7A-D depict an embodiment of co-staining of intestinal markers (red), human CD45 (green) and human CDHI (white) on sections of HIOs at 12, 16 and 20 weeks post transplantation in humanized mice. Images represent presence of enterocytes (VIL/Villin) (FIG. 7A), goblet cells (MUC2/Mucin2) (FIG. 7B), Paneth cells (LYZ/Lysozyme) (FIG. 7C) and enteroendocrine cells (CHGA/Chromogranin A) (FIG. 7D). Scale bar represents 100 μm.

FIG. 8 depicts an embodiment of UMAP graphs represent the expression of each marker across the samples.

FIG. 9 depicts an embodiment of a heatmap graph representing the level of expression of each markers per cell. Each bar on the heatmap corresponds to a cell from a sample (HIO or mouse small intestine (SI) at 12, 16 or 20 weeks post transplantation). Top x axis corresponds to the cluster/identified cell type. Bottom x axis corresponds to the group: HIO or mouse small intestine (SI) at 12, 16 or 20 weeks (see color legend on top right corner).

FIG. 10A depicts an embodiment of contour plots illustrate the graphs (bottom) representing the expression of TNF-α, IFN-γ and IL-2 in unstimulated or PMA/ionomycin (PMA/iono)-treated human CD4+ T cells isolated from HIO (black square; n=4 samples) or mouse small intestine (SI) (open square; n=4 samples) from humanized mice at 16 weeks post transplantation. Mean+/−SD. Wilcoxon matched-pairs signed rank test. Non-significant (n.s).

FIG. 10B depicts an embodiment of indicated cytokines were measured by multiplex (Luminex®) assay in supernatants of immune cells isolated from HIO (black square; n=3 samples) or mouse small intestine (SI) (open square; n=3 samples) of humanized mice at 16 weeks post transplantation and treated for 3 days with anti-CD3/CD28 antibody or untreated media. Mean+/−SD. Wilcoxon matched-pairs signed rank test. Non-significant (n. s).

FIGS. 11A-B depicts an embodiment of humanized mouse small intestine at 12, 16 and 20 weeks post HIO transplantation, were stained with anti-human CD3 (T cells) (FIG. 11A) or CD20 (B cells) (FIG. 11B) by immunohistochemistry(IHC) and counterstained with hematoxylin. In panel (FIG. 11B) black arrowheads indicate presence of B cells in humanized mouse small intestine. Scale bar represents 50 μm.

FIG. 12 depicts an embodiment of schema summarizing the cellular mechanism of lymphoid follicle formation during fetal gut development described in the literature. Briefly, around 11 post conceptual weeks (PCW), T cells start to invade the gut followed by B cells. Around 14 to 16 PCW, T and B cells form aggregates and later mature into lymphoid follicles, at 19 PCW. Plasma cells as well as granulocytes were observed in fetal intestine at 22 PCW.

FIG. 13A depicts an embodiment of enteroid monolayers were grown to confluence, differentiated for 5 days (with DF or M cell media), immunostained with glycoprotein 2 (GP-2) and actin and imaged by confocal microscopy.

FIG. 13B depicts an embodiment of enteroid monolayers grown in M cell media and stained with GP-2 and imaged by confocal microscopy.

FIG. 13C depicts an embodiment of qPCR for M cell specific transcription factors SOX8 and SPI-B as well as mature M cell marker GP2. Mean+/−SEM.

FIG. 14A depicts an embodiment of transplanted HIO at 12 weeks with mouse kidney seen underneath from control or humanized mice.

FIG. 14B depicts an embodiment of a graph representing the length of HIOs from control (black dot) or humanized (green square) group. n=5 control and n=4 humanized.

FIG. 14C depicts an embodiment of a graph representing immunophenotyping of peripheral blood from humanized mice at 22 weeks post UCB engraftment or 12 weeks post HIO transplantation.

FIGS. 14D-F depicts an embodiment of formalin-fixed paraffin-embedded (FFPE) sections of transplanted HIO at 12 weeks H&E-stained (FIG. 14D) or stained by IHC with anti-human CD45 (FIG. 14E) and anti-human CD3 and CD20 antibodies (FIG. 14F). Scale bar represents 100 μm.

FIGS. 15A-B depicts an embodiment of graphs represent gene expression of indicated chemokines in transplanted HIO (FIG. 15A) or humanized mouse small intestine (SI) (FIG. 15B) at 16 weeks. n=3 HIO and n=3 humanized mouse SI. Gene expression is normalized to housekeeping gene (GAPDH). Mean+/− standard deviation (SD).

### DETAILED DESCRIPTION

Although experimental mouse models have been widely used to dissect the biological mechanisms of intestinal immune tissue development and disease, there are still substantial differences that cannot be extrapolated to human biology. The methods currently used to dissect the crosstalk between human immune and intestinal cells are based on in vitro cultures of induced-pluripotent stem cell-or tissue-derived organoids with immune cells or its derivatives and do not fully recapitulate the complexity of the intestinal tissue. Better understanding of the perturbations of the GI immune system that commonly result in chronic human diseases will benefit from new human models that represent patient specific GI immune tissue.

Embodiments disclosed herein include a novel humanized model with HIO engraftment resulted in the development of functional immune aggregates and epithelial differentiation of M cells. This novel and unique model can be used as a tool to investigate human intestinal organogenesis and develop new therapies to treat GI diseases.

A key feature of the pluripotent stem cells (PSC)-derived HIO model is to generate a complex human intestinal tissue. To address the lack of circulating immune cells due to the immunosuppression present in the murine model, in an embodiment disclosed herein an in vivo HIO with immune cellular features using humanized immune system mice was introduced as a support for human hematopoietic cells as well as HIO development.

Humanized immune system mouse models are commonly used to investigate human hematopoiesis or inflammatory diseases and can be generated by the engraftment of human peripheral blood leukocytes, hematopoietic stem cells or fetal tissues (bone marrow, thymus and/or liver) in lymphopenic mice such as NOD/SCID/112rg−/− (NSG) mouse strain. It was reported that transgenic expression of human SCF, GM-CSF and IL-3 in NSG mice, named NSGS mice, improves hematopoietic engraftment, reconstitution, and function. However, because these lymphopenic mice do not express Il2rg, resulting in impaired signaling in lymphoid tissue inducer (LTi) cells, they do not have Peyer's patches and lymphoid follicles in their intestine.

In an embodiment disclosed herein, a serial time course following transplantation of HIO under the kidney capsule was performed characterizing human immune cells infiltrating the HIO lamina propria and epithelium. Mass cytometry and immunostaining confirmed the presence of GALT-associated B cells in cellular immune aggregates present in transplanted HIOs resembling lymphoid follicles developing in human fetal intestine. It was demonstrated that the administration of microbial lysate in the HIO lumen induces the epithelial expression of GP2 at the cell surface of M cells. To determine whether M cells were functional in translocating luminal antigen to activate of immune cells in the lamina propria, the response of plasma cells was evaluated and there was an increase of secreted IgA antibodies in the HIO lumen. Altogether, these results demonstrate that crosstalk between the HIO epithelium and the immune cells induces the formation of lymphoid-like structures and M cell differentiation and function.

### Terms

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The disclosure herein uses affirmative language to describe the numerous embodiments. The disclosure also includes embodiments in which subject matter is excluded, in full or in part, such as substances or materials, method steps and conditions, protocols, or procedures.

The articles “a” and “an” are used herein to refer to one or to more than one (for example, at least one) of the grammatical object of the article. By way of example, “an element”means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of”′ is meant including, and limited to, whatever follows the phrase “consisting of.” Thus, the phrase “consisting of” indicates that the listed elements are required or mandatory, and that no other elements may be present. By “consisting essentially of”′ is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases, The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity can be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield can be affected by the efficiency of a reaction or process, unwanted side reactions, degradation, quality of the input substances, compounds, or materials, or loss of the desired substance, compound, or material during any step of the production.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Cryoprotectants are cell composition additives to improve efficiency and yield of low temperature cryopreservation by preventing formation of large ice crystals. Cryoprotectants include but are not limited to DMSO, ethylene glycol, glycerol, propylene glycol, trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. Cryoprotectants can be used as part of a cryopreservation medium, which include other components such as nutrients (e.g. albumin, serum, bovine serum, fetal calf serum [FCS]) to enhance post-thawing survivability of the cells. In these cryopreservation media, at least one cryoprotectant may be found at a concentration that is, is about, is at least, is at least about, is not more than, or is not more than about, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any percentage within a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, B-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “basement membrane matrix” or “extracellular matrix” as used herein has its plain and ordinary meaning in light of the specification and refers to any biological or synthetic compound, substance, or composition that enhances cell attachment and/or growth. Any extracellular matrix, as well as any mimetic or derivative thereof, known in the art can be used for the methods disclosed herein. Some examples of extracellular matrices, or mimetics or derivative thereof, include but are not limited to cell-based feeder layers, polymers, proteins, polypeptides, nucleic acids, sugars, lipids, poly-lysine, poly-ornithine, collagen, collagen IV, gelatin, fibronectin, vitronectin, laminin, laminin-511 elastin, tenascin, heparan sulfate, entactin, nidogen, osteopontin, perlecan, basement membrane, Matrigel, hydrogel, PEI, WGA, or hyaluronic acid, or any combination thereof. A common basement membrane matrix that is used in laboratories are those isolated from murine Engelbreth-Holm-Swarm (EHS) sarcoma cells. However, these basement membrane matrices are derived from non-human animals and therefore contain xenogeneic components that prevent its use towards humans. They are also not defined, which can lead to variability in manufacturing, as well as potentially harbor pathogens. Accordingly, in some embodiments, the methods for culturing cells may involve the use of synthetic and/or defined alternatives to these xenogeneic basement membrane matrices. The use of non-xenogeneic basement membrane matrices or mimetics or derivatives thereof enables manufacturing of biological products better suited for human use.

The terms “passage” and “passaging” as used herein have their plain and ordinary meaning as understood in light of the specification, and refer to the conventional approaches performed in biological cell culture methods to maintain a viable population of cells for prolonged periods of time. As cells are generally proliferative in cell culture, they undergo multiple cycles of mitosis until occupying the available space, which is typically a surface of a cell culture container (e.g., a plate, dish, or flask) submerged under culture medium. For example, the cells may grow out as a monolayer on a cell culture container surface. If the growing cells occupy the entire available space of surface, they cannot proliferate further and may exhibit senescent behavior. In order to continue growth of the cells, which may be performed to maintain the viability and proliferative nature of the cells and/or to expand the number of cells for downstream purposes, the cells may be passaged by taking a fraction of the cells and seeding this fraction onto a fresh surface (e.g., of a cell culture container) in culture medium. This fraction of the cells will continue to proliferate and multiply until they occupy the available space of the new surface, upon which this passaging can be repeated successively.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

### Stem Cells

The term “totipotent stem cells” (also known as omnipotent stem cells) as used herein has its plain and ordinary meaning as understood in light of the specification and are stem cells that can differentiate into embryonic and extra-embryonic cell types. Such cells can construct a complete, viable organism. These cells are produced from the fusion of an egg and sperm cell. Cells produced by the first few divisions of the fertilized egg are also totipotent.

The term “embryonic stem cells (ESCs),” also commonly abbreviated as ES cells, as used herein has its plain and ordinary meaning as understood in light of the specification and refers to cells that are pluripotent and derived from the inner cell mass of the blastocyst, an early-stage embryo. For purpose of the present disclosure, the term “ESCs” is used broadly sometimes to encompass the embryonic germ cells as well.

The term “pluripotent stem cells (PSCs)” as used herein has its plain and ordinary meaning as understood in light of the specification and encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

The term “induced pluripotent stem cells (IPSCs),” also commonly abbreviated as iPS cells, as used herein has its plain and ordinary meaning as understood in light of the specification and refers to a type of pluripotent stem cells artificially derived from a normally non-pluripotent cell, such as an adult somatic cell, by inducing a “forced” expression of certain genes. hiPSC refers to human iPSCs. In some methods known in the art, iPSCs may be derived by transfection of certain stem cell-associated genes into non-pluripotent cells, such as adult fibroblasts. Transfection may be achieved through viral transduction using viruses such as retroviruses or lentiviruses. Transfected genes may include the master transcriptional regulators Oct-3/4 (POUSF1) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some methods, a retroviral system is used to transform human fibroblasts into pluripotent stem cells using four pivotal genes: Oct3/4, Sox2, Klf4, and c-Myc. In other methods, a lentiviral system is used to transform somatic cells with OCT4, SOX2, NANOG, and LIN28. Genes whose expression are induced in iPSCs include but are not limited to Oct-¾ (POU5F1); certain members of the Sox gene family (e.g., Sox1 Sox2, Sox3, and Sox15); certain members of the Klf family (e.g., Klf1, Klf2, Klf4, and Klf5), certain members of the Myc family (e.g., C-myc, L-myc, and N-myc), Nanog, LIN28, Tert, Fbx15, ERas, ECAT15-1, ECAT15-2, Tel1, β-Catenin, ECAT1, Esg1, Dnmt3L, ECAT8, Gdf3, Fth117, Sal14, Rex1, UTF1, Stella, Stat3, Grb2, Prdm14, Nr5a1, Nr5a2, or E-cadherin, or any combination thereof.

The term “precursor cell” as used herein has its plain and ordinary meaning as understood in light of the specification and encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), and epiblast stem cells (EpiSC).

In some embodiments, one step is to obtain stem cells that are pluripotent or can be induced to become pluripotent. In some embodiments, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. It would be understood by one of skill in the art that the methods and systems described herein are applicable to any stem cells.

Additional stem cells that can be used in embodiments in accordance with the present disclosure include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Exemplary embryonic stem cells that can be used in embodiments in accordance with the present disclosure include but are not limited to SA01 (SA001); SA02 (SA002); ES01 (HES-1); ES02(HES-2); ES03(HES-3); ES04(HES-4); ES05(HES-5); ES06 (HES-6); BG01 (BGN-01); BG02 (BGN-02); BG03 (BGN-03); TE03(13); TE04(14); TE06 (16); UCO1 (HSF1); UC06 (HSF6); WA01 (HI); WA07 (H7); WA09 (H9); WA13 (H13); WA14 (H14). Exemplary human pluripotent cell lines include but are not limited to TkDA3-4, 1231A3, 317-D6, 317-A4, CDH1, 5-T-3, 3-34-1, NAFLD27, NAFLD77, NAFLD150, WD90, WD91, WD92, L20012, C213, 1383D6, FF, or 317-12 cells.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

In some embodiments, an adenovirus can be used to transport the requisite four genes, resulting in iPSCs substantially identical to embryonic stem cells. Since the adenovirus does not combine any of its own genes with the targeted host, the danger of creating tumors is eliminated. In some embodiments, non-viral based technologies are employed to generate iPSCs. In some embodiments, reprogramming can be accomplished via plasmid without any virus transfection system at all, although at very low efficiencies. In other embodiments, direct delivery of proteins is used to generate iPSCs, thus eliminating the need for viruses or genetic modification. In some embodiment, generation of mouse iPSCs is possible using a similar methodology: a repeated treatment of the cells with certain proteins channeled into the cells via poly-arginine anchors was sufficient to induce pluripotency. In some embodiments, the expression of pluripotency induction genes can also be increased by treating somatic cells with FGF2 under low oxygen conditions.

The term “feeder cell” as used herein has its plain and ordinary meaning as understood in light of the specification and refers to cells that support the growth of pluripotent stem cells, such as by secreting growth factors into the medium or displaying on the cell surface. Feeder cells are generally adherent cells and may be growth arrested. For example, feeder cells are growth-arrested by irradiation (e.g. gamma rays), mitomycin-C treatment, electric pulses, or mild chemical fixation (e.g. with formaldehyde or glutaraldehyde). However, feeder cells do not necessarily have to be growth arrested. Feeder cells may serve purposes such as secreting growth factors, displaying growth factors on the cell surface, detoxifying the culture medium, or synthesizing extracellular matrix proteins. In some embodiments, the feeder cells are allogeneic or xenogeneic to the supported target stem cell, which may have implications in downstream applications. In some embodiments, the feeder cells are mouse cells. In some embodiments, the feeder cells are human cells. In some embodiments, the feeder cells are mouse fibroblasts, mouse embryonic fibroblasts, mouse STO cells, mouse 3T3 cells, mouse SNL 76/7 cells, human fibroblasts, human foreskin fibroblasts, human dermal fibroblasts, human adipose mesenchymal cells, human bone marrow mesenchymal cells, human amniotic mesenchymal cells, human amniotic epithelial cells, human umbilical cord mesenchymal cells, human fetal muscle cells, human fetal fibroblasts, or human adult fallopian tube epithelial cells. In some embodiments, conditioned medium prepared from feeder cells is used in lieu of feeder cell co-culture or in combination with feeder cell co-culture. In some embodiments, feeder cells are not used during the proliferation of the target stem cells.

### Differentiation of PSCs

Known methods for making downstream cell types, such as definitive endoderm, foregut endoderm, ventral foregut endoderm, and hepatic lineages from pluripotent cells (e.g., iPSCs or ESCs) are applicable to the methods described herein. In some embodiments, pluripotent cells are derived from a morula. In some embodiments, pluripotent stem cells are stem cells. Stem cells used in these methods can include, but are not limited to, embryonic stem cells or induced pluripotent stem cells. Embryonic stem cells can be derived from the embryonic inner cell mass or from the embryonic gonadal ridges. Embryonic stem cells can originate from a variety of animal species including, but not limited to, various mammalian species including humans. In some embodiments, human embryonic stem cells are used to produce definitive endoderm or other downstream cell types such as foregut endoderm, ventral foregut endoderm, and hepatic lineages. In some embodiments, iPSCs are used to produce definitive endoderm or other downstream cell types such as foregut endoderm, ventral foregut endoderm, and hepatic lineages. In some embodiments, human iPSCs (hiPSCs) are used to produce definitive endoderm or other downstream cell types such as foregut endoderm, ventral foregut endoderm, and hepatic lineages.

In some embodiments, PSCs, such as ESCs and iPSCs, undergo directed differentiation into embryonic germ layer cells, organ tissue progenitor cells, and then into tissue such as liver tissue or any other biological tissue. In some embodiments, the directed differentiation is done in a stepwise manner to obtain each of the differentiated cell types where molecules (e.g. growth factors, ligands, agonists, antagonists) are added sequentially as differentiation progresses. In some embodiments, the directed differentiation is done in a non-stepwise manner where molecules (e.g. growth factors, ligands, agonists, antagonists) are added at the same time. In some embodiments, directed differentiation is achieved by selectively activating certain signaling pathways in the PSCs or any downstream cells.

In some embodiments, the embryonic stem cells or iPSCs are treated with one or more small molecule compounds, activators, inhibitors, or growth factors for a time that is, is about, is at least, is at least about, is not more than, or is not more than about, 6 hours, 12 hours, 18 hours, 24 hours, 36 hours, 48 hours, 60 hours, 72 hours, 84 hours, 96 hours, 120 hours, 150 hours, 180 hours, 240 hours, 300 hours or any time within a range defined by any two of the aforementioned times, for example 6 hours to 300 hours, 24 hours to 120 hours, 48 hours to 96 hours, 6 hours to 72 hours, or 24 hours to 300 hours. In some embodiments, more than one small molecule compounds, activators, inhibitors, or growth factors are added. In these cases, the more than one small molecule compounds, activators, inhibitors, or growth factors can be added simultaneously or separately.

In some embodiments, the embryonic stem cells or iPSCs are treated with one or more small molecule compounds, activators, inhibitors, or growth factors at a concentration that is, is about, is at least, is at least about, is not more than, or is not more than about, 10 ng/ml, 20 ng/ml, 50 ng/ml, 75 ng/ml, 100 ng/ml, 120 ng/ml, 150 ng/ml, 200 ng/ml, 500 ng/ml, 1000 ng/ml, 1200 ng/ml, 1500 ng/mL, 2000 ng/ml, 5000 ng/ml, 7000 ng/ml, 10000 ng/ml, or 15000 ng/ml, or any concentration that is within a range defined by any two of the aforementioned concentrations, for example, 10 ng/mL to 15000 ng/ml, 100 ng/ml to 5000 ng/ml, 500 ng/ml to 2000 ng/ml, 10 ng/ml to 2000 ng/ml, or 1000 ng/ml to 15000 ng/mL. In some embodiments, concentration of the one or more small molecule compounds, activators, inhibitors, or growth factors is maintained at a constant level throughout the treatment. In some embodiments, concentration of the one or more small molecule compounds, activators, inhibitors, or growth factors is varied during the course of the treatment. In some embodiments, more than one small molecule compounds, activators, inhibitors, or growth factors are added. In these cases, the more than one small molecule compounds, activators, inhibitors, or growth factors can differ in concentrations.

In some embodiments, the ESCs or iPSCs are cultured in growth media that supports the growth of stem cells. In some embodiments, the ESCs or iPSCs are cultured in stem cell growth media. In some embodiments, the stem cell growth media is RPMI 1640, DMEM, DMEM/F12, or Advanced DMEM/F12. In some embodiments, the stem cell growth media comprises fetal bovine serum (FBS). In some embodiments, the stem cell growth media comprises FBS at a concentration that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20%, or any percentage within a range defined by any two of the aforementioned concentrations, for example 0% to 20%, 0.2% to 10%, 2% to 5%, 0% to 5%, or 2% to 20%. In some embodiments, the stem cell growth media does not contain xenogeneic components. In some embodiments, the growth media comprises one or more small molecule compounds, activators, inhibitors, or growth factors.

In some embodiments, pluripotent stem cells are prepared from somatic cells. In some embodiments, pluripotent stem cells are prepared from biological tissue obtained from a biopsy. In some embodiments, the pluripotent stem cells are cryopreserved. In some embodiments, the somatic cells are cryopreserved. In some embodiments, pluripotent stem cells are prepared from PBMCs. In some embodiments, human PSCs are prepared from human PBMCs. In some embodiments, pluripotent stem cells are prepared from cryopreserved PBMCs. In some embodiments, PBMCs are grown on a feeder cell substrate. In some embodiments, PBMCs are grown on a mouse embryonic fibroblast (MEF) feeder cell substrate. In some embodiments, PBMCs are grown on an irradiated MEF feeder cell substrate.

In some embodiments, stem cells are treated with one or more growth factors to differentiate to definitive endoderm cells. Such growth factors can include growth factors from the TGF-beta superfamily. In some embodiments, the one or more growth factors comprise the Nodal/Activin and/or the BMP subgroups of the TGF-beta superfamily of growth factors. In some embodiments, the one or more growth factors are selected from the group consisting of Nodal, Activin A, Activin B, BMP4, Wnt3a or combinations of any of these growth factors. In some embodiments, the stem cells are contacted with Activin A. In some embodiments, the stem cells are contacted with Activin A and BMP4.

In some embodiments, definitive endoderm (DE) can further undergo anterior endoderm pattering, foregut specification and morphogenesis, dependent on FGF, Wnt, BMP, or retinoic acid, or any combination thereof. In some embodiments, human PSCs are efficiently directed to differentiate in vitro into liver epithelium and mesenchyme. It will be understood that molecules such as growth factors can be added to any stage of the development to promote a particular type of hepatic tissue formation. In some embodiments, siRNA and/or shRNA targeting cellular constituents associated with the FGF, Wnt, BMP, or retinoic acid signaling pathways are used to inhibit or activate these pathways.

### Intestinal Organoids and Methods of Making

The term “intestinal organoid” as used herein has its plain and ordinary meaning as understood in light of the specification and refers to three-dimensional cellular structures that present many properties of the small intestine of an organism. In some embodiments, intestinal organoids relate to those derived from human cells and exhibit the properties of a human small intestine. However, intestinal organoids from other mammals are also encompassed. Intestinal organoids as used herein are derived from pluripotent stem cells (e.g. embryonic stem cells or induced pluripotent stem cells) or an intermediate thereof (e.g. definitive endoderm), where the process of differentiating pluripotent stem cells into definitive endoderm, then hindgut endoderm (which may be in the form of spheroids), and finally to an intestinal organoid results in a cellular structure that has the composition, structure, and function resembling a naturally developed intestine. A significant difference between the intestinal organoids used herein and are cellular structures derived from adult intestinal epithelium, sometimes referred to as “enteroids”, and other so-called organoids produced from non-pluripotent adult intestinal stem cells, is that the intestinal organoids used herein contain both epithelium and mesenchyme. The mesenchyme performs an important supportive role for the epithelium, and greatly enhances the viability and robust function of the intestinal organoid. The intestinal organoids used herein may exhibit a lumen with epithelial villus-like involutions closely resembling normal intestine, and peristaltic behavior. As a result of the differentiation process from pluripotent stem cells, the intestinal organoids used herein also contain specialized intestinal cell types, including enterocytes, Goblet cells, Paneth cells, and enteroendocrine cells. References disclosing embodiments of intestinal organoids suitable for use herein include U.S. Pat. Nos. 9,719,068 and 10,781,425, U.S. Patent Application Publication US 2020/190478, and PCT Publication WO 2011/140441, WO 2016/061464, WO 2018/106628, WO 2018/200481, WO 2019/126626, WO 2020/160371, WO 2020/056158, WO 2020/243633, and WO 2021/030373, each of which are incorporated herein by reference in their entirety.

The term “mucosa” as used herein has its plain and ordinary meaning as understood in light of the specification and refers to the most inner layer of the gastrointestinal tract. The epithelium is the most inner layer of the mucosa, and is where epithelial cells and other specialized cells such as Goblet cells are found. The epithelium also forms the villi structure of the intestine. The epithelium is surrounded by connective tissue called the lamina propria, and a thin layer of smooth muscle.

The term “muscularis” as used herein has its plain and ordinary meaning as understood in light of the specification and refers to the muscularis propria of the gastrointestinal tract. The muscularis regulates peristaltic behavior of the intestine and colon, and originates from the mesenchymal layer of the nascent gut tube during development.

The term “intestinal barrier” as used herein has its plain and ordinary meaning as understood in light of the specification and refers to the cellular and mucosal barrier that separates the intraluminal contents of the gastrointestinal tract from the surrounding tissue and circulatory system, while still permitting nutrient exchange. This barrier is mediated by the intracellular junctions between the cells of the epithelium. During intestinal damage, this barrier can be disrupted, resulting in abnormal function of the intestine, passage of potentially pathogenic microorganisms or antigens into the body, and leaking of blood and molecules into the lumen.

The intestinal organoids disclosed herein are produced by a differentiation process from pluripotent stem cells (such as embryonic stem cells or induced pluripotent stem cells) or an intermediate thereof (such as definitive endoderm), and comprise epithelial cell types and mesenchymal cell types, along with intestinal or colonic specialized cell types. Exemplary methods for making intestinal organoids can be found in U.S. Pat. Nos. 9,719,068, and 10,781,425, U.S. Patent Application Publication US 2020/190478 and PCT Publications WO 2011/140441, WO 2016/061464, WO 2018/106628, WO 2018/200481, WO 2019/126626, WO 2020/056158, WO 2020/160371, WO 2020/243633, WO 2021/030373, each of which is hereby expressly incorporated by reference in its entirety.

In some embodiments, intestinal organoids are differentiated through the culture of definitive endoderm cells. These definitive endoderm cells can be differentiated from pluripotent cells by contacting the definitive endoderm with the Nodal, Activin, and/or BMP subgroups of the TGFβ superfamily of growth factors. In some embodiments, the pluripotent stem cells are contacted with Nodal, Activin A, Activin B, BMP4, or any combination thereof, to differentiate the pluripotent stem cells to definitive endoderm. In some embodiments, the pluripotent stem cells are contacted with Activin A to differentiate the pluripotent stem cells to definitive endoderm.

Definitive endoderm can further be subjected to FGF/Wnt-induced posterior endoderm patterning to direct hindgut specification.

In some embodiments, to produce intestinal organoids, definitive endoderm is first contacted with a Wnt signaling pathway activator and an FGF signaling pathway activator to posteriorize the definitive endoderm to hindgut endoderm. During this culture process, hindgut endoderm grows as monolayer but also spontaneously buds off as clumps of cells called hindgut spheroids in suspension. In some embodiments, the Wnt signaling pathway activator comprises Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, or Wnt16, or any combination thereof. In some embodiments, the Wnt signaling pathway activator is Wnt3a. In some embodiments, the Wnt signaling pathway activator comprises a glycogen synthase kinase-3 (GSK3) inhibitor, which acts as a Wnt signaling pathway activator. In some embodiments, the GSK3 inhibitor is CHIR99021. In some embodiments, the FGF signaling pathway activator comprises FGF1, FGF2, FGF3, FGF4, FGF4, FGF5, FGF6, FGF7, FGF8, FGF8, FGF9, FGF10, FGF11, FGF12, FGF13, FGF14, FGF15 (FGF19, FGF15/FGF19), FGF16, FGF17, FGF18, FGF20, FGF21, FGF22, FGF23, or any combination thereof. In some embodiments, the FGF signaling pathway activator is FGF4. The hindgut endoderm and hindgut spheroids produced comprise CDX2+ polarized epithelium surrounded by CDX2+ mesenchyme, and lack Alb and Pdx1, which denote foregut endoderm.

Following formation of hindgut endoderm, or hindgut spheroids, which can be manipulated in suspension and embedded in a basement membrane matrix (e.g. Matrigel) for three-dimension culture, the BMP signaling pathway regulates formation of distinct regional types of intestine. Inhibition of BMP signaling after the hindgut stage promotes a proximal intestinal fate (duodenum/jejunum). Activation of BMP signaling after the hindgut stage promotes a more distal intestinal cell fate (cecum/colon). In some embodiments, the hindgut endoderm is contacted with a BMP signaling pathway activator to differentiate the hindgut endoderm into an intestinal organoid. In some embodiments, the hindgut endoderm is contacted with a BMP signaling pathway inhibitor to differentiate the hindgut endoderm into a colonic organoid. In some embodiments, the BMP signaling pathway activator comprises BMP1, BMP2, BMP3, BMP4, BMP5, BMP6, BMP7, BMP8a, BMP8b, BMP10, BMP11, BMP15, IDE1, or IDE2, or any combination thereof. In some embodiments, the BMP signaling pathway activator comprises BMP2. In some embodiments, the BMP signaling pathway inhibitor comprises Noggin, RepSox, LY364947, LDN193189, or SB431542, or any combination thereof. In some embodiments, the BMP signaling pathway inhibitor comprises Noggin.

### Methods of Generating Intestinal Organoids With Immune Cells

Methods of generating an intestinal organoid (IO) with immune cells (iIO) are disclosed herein. In some embodiments, the IO is transplanted into an organism having immune cells for a period of time sufficient to form an IO having immune cells in one or more cell layers of the IO (iIO). In some embodiments, the period of time is, is about, is at least, or is at least about, 6, 8, 10, 12, 14, 16, 18, or 20 weeks, or a range defined by any two of the preceding values, optionally, 6-20, 12-20 or 16-20 weeks. In some embodiments, the period of time is, is about, is at least, or is at least about, 12 weeks or 16 weeks.

In some embodiments, the iIO comprises CD4+ T cells and/or CD20+ B cells localized to lamina propria and epithelium. In some embodiments, the iIO comprises aggregates of T and B cells. In some embodiments, the iIO comprises CD4+ T cells and CD8+ T cells in a T-cell zone. The term “cell zone” and “cellular zonation” as used herein has its plain and ordinary meaning as understood in light of the specification and refers to the colocalization of cells of the same type. In some embodiments, the iIO comprises plasma cells and neutrophils.

In some embodiments, the iIO expresses gut-associated lymphoid tissue (GALT)-associated chemokines. In some embodiments, the GALT-associated chemokines are CCL19, CCL21, and/or CXCL13. In some embodiments, the iIO comprises GALT-associated B cells. In some embodiments, the B cells are aggregated in a lymphoid-like structure. In some embodiments, the iIO comprises CD45+ cells. In some embodiments, the CD45+ cells are localized to a mucosal layer, lamina propria, and/or epithelium of the iIO. In some embodiments, the CD45+ cells form cellular aggregates.

In some embodiments, the iIO comprises CD3+ B cells and CD20+ T cells. In some embodiments, the iIO comprises CD4+ T cells, innate lymphoid cells, mucosal-associated invariant T (MAIT)-like cells, CD8+ T cells, lymphoid tissue inducer-like cells, B cells, natural killer cells, dendritic cells, intraepithelial lymphocytes, macrophages, natural killer T cells, and/or neutrophils. In some embodiments, the iIO comprises enterocytes, goblet cells, Paneth cells, and enteroendocrine cells. In some embodiments, the iIO comprises Villin+, Mucin2+, Lysozyme+, and/or Chromogranin A+ cells.

In some embodiments, upon exposure of the lumen of the iIO to a lysate of Escherichia coli, microfold cells (M cells) of the iIO express glycoprotein 2 (GP2) at the cell surface. In some embodiments, upon exposure of the lumen of the iIO to a lysate of Escherichia coli, plasma cells of the iIO produce IgA antibodies. In some embodiments, upon exposure of the lumen of the iIO to a lysate of Escherichia coli, M cells and B cells of the iIO are co-localized.

In some embodiments, the organism is not human. In some embodiments, the IO is transplanted into a kidney capsule of the organism. In some embodiments, the organism does not have Peyer's patches or lymphoid follicles in an intestine. In some embodiments, the organism is a mouse. In some embodiments, the mouse is a lymphopenic mouse. In some embodiments, the mouse is a NOD/SCID/Il2rg−/− mouse with transgenic expression of human SCF, GM-CSF, and IL-3 (NSGS mouse). In some embodiments, prior to transplanting of the IO, the mouse is injected with cord blood cells, optionally wherein the cord blood cells are human cord blood cells. In some embodiments, the mouse is treated with a pharmaceutical compound prior to the injection of cord blood cells to optimize engraftment of cord blood cells, for example, a pharmaceutical compound (e.g., a chemotherapy drug such as busulfan) that depletes mouse bone marrow cells.

In some embodiments, the IO is made by a method comprising a) exposing definitive endoderm (DE) cells to an FGF activator and a Wnt pathway activator for a period of time to differentiate into a hindgut spheroid, for example, a mid-hindgut spheroid; b) embedding the hindgut spheroid into a basement membrane matrix; and c) exposing the embedded hindgut spheroid to EGF for a period of time to differentiate into an IO, optionally wherein the IO is a human IO (HIO). In some embodiments, the IO is made by a method known in the art, or by a method disclosed herein.

Also disclosed herein are methods of generating an intestinal organoid (IO) with immune cells (iIO), wherein the method further comprises, prior to implanting the IO, making the IO. In some embodiments, the method of making the IO comprises a) exposing definitive endoderm (DE) cells to an FGF activator and a Wnt pathway activator for a period of time to differentiate into a mid-hindgut spheroid, for example, a mid-hindgut spheroid; b) embedding the hindgut spheroid into a basement membrane matrix; and c) exposing the embedded hindgut spheroid to EGF for a period of time to differentiate into an IO, optionally wherein the IO is an HIO. In some embodiments, the IO is made by a method known in the art, or by a method disclosed herein.

In some embodiments, the DE cells are derived from pluripotent stem cells (PSCs), optionally wherein the PSCs are embryonic stem cells (ESCs) or induced pluripotent stem cells (iPSCs), optionally wherein the PSCs are human PSCs, as disclosed herein. In some embodiments, the basement membrane matrix environment is Matrigel.

In some embodiments, the FGF activator is FGF4, optionally wherein the concentration is, is about, is at least, or is at least about 50 ng/ml, 100 ng/ml, 150 ng/ml, 200 ng/ml, 250 ng/ml, 300 ng/ml, 350 ng/ml, 400 ng/ml, 450 ng/ml, 500 ng/ml, 550 ng/ml, 600 ng/ml, 650 ng/ml, 700 ng/ml, or 750 ng/ml, or a range defined by any two of the preceding values, optionally 50 -750 ng/ml, 50-100 ng/ml, or 50-500 ng/ml, or optionally at a concentration of 500 ng/ml. In some embodiments, the Wnt pathway activator is CHIRON 99021, optionally wherein the concentration is, is about, is at least, or is at least about 0.5 μM, 1 μM, 1.5 μM, 2 μM, 2.5 μM, 3 μM, 3.5 μM, 4 μM, 4.5 μM, 5 μM, 5.5 M, or 6 μM, or a range defined by any two of the preceding values, optionally 0.5-6 μM, 0.5-3 μM, 3-6 μM, 2-4 μM, or optionally at a concentration of 3 μM. In some embodiments, the concentration of EGF is, is about, is at least, or is at least about 25 ng/ml, 50 ng/ml, 75 ng/ml, 100 ng/ml, 125 ng/ml, 150 ng/ml, 175 ng/ml, or 200 ng/ml, or a range defined by any two of the preceding values, optionally 25-100 ng/ml, 50 -150 ng/ml, 100 ng/ml, or optionally is at a concentration of 100 ng/ml. In some embodiments, the IO is matured in vitro for a period of time prior to transplantation, optionally wherein the period of time is, is about, is at least, or is at least about, 7, 10, 14, 16, 21, 25, or 28 days, or a range defined by any two of the preceding values, optionally 7-28, 14-28, or 21-28 days.

### Methods of Generating Intestinal Organoids With Activated Immune Cells

Also disclosed herein are methods of making an iIO with activated immune cells. In some embodiments, an immune stimulating material, for example, an allergen, and/or microbial lysate, is administered to a lumen of the iIO. In some embodiments, the iIO is made by the methods disclosed herein. In some embodiments, the microbial lysate comprises Escherichia coli. In some embodiments, the iIO with activated immune cells comprises GP2+ microfold (M) cells after a period of time post-administration of the immune stimulating material, e.g., allergen, and/or microbial lysate. In some embodiments, the period of time post-administration is, is about, is at least, or is at least about, 24, 36, 48, 60, or 72 hours. In some embodiments, the mucus of the iIO with activated immune cells comprises secreted IgA antibodies. In some embodiments, M cells and B cells colocalize within the iIO with activated immune cells. In some embodiments, the iIO with activated immune cells made by methods disclosed herein.

### Methods of Generating Human Intestinal Organoids With Human/humanized Immune Cells

In some embodiments, the IO is a human IO (HIO). In some embodiments, the immune cells of the organism comprise human immune cells, and/or the organism has a humanized immune system. In some embodiments, the IO is an HIO, wherein the organism has human immune cells and/or a humanized immune system, and the HIO comprises human immune cells and/or humanized immune cells in one or more layers of the HIO (hiHIO).

### Intestinal Organoid Compositions Having Immune Cells

Disclosed herein are intestinal organoids (IO) comprising immune cells in one or more cell layers of the IO (iIO). In some embodiments, the iIO is made by any method disclosed herein. In some embodiments, the iIO comprises CD4+ T cells and/or CD20+ B cells localized to lamina propria and epithelium. In some embodiments, the iIO comprises aggregates of T and B cells. In some embodiments, the iIO comprises CD4+ T cells and CD8+ T cells in a T-cell zone. In some embodiments, the iIO comprises plasma cells and neutrophils. In some embodiments, the iIO expresses gut-associated lymphoid tissue (GALT)-associated chemokines. In some embodiments, the GALT-associated chemokines are CCL19, CCL21, and/or CXCL13. In some embodiments, the iIO comprises GALT-associated B cells. In some embodiments, the B cells are aggregated in a lymphoid-like structure. In some embodiments, the iIO comprises CD45+ cells. In some embodiments, the CD45+ cells are localized to a mucosal layer, lamina propria, and/or epithelium of the iIO. In some embodiments, the CD45+ cells form cellular aggregates. In some embodiments, the iIO comprises CD3+ B cells and CD20+ T cells. In some embodiments, the iIO comprises CD4+ T cells, innate lymphoid cells, mucosal-associated invariant T (MAIT)-like cells, CD8+ T cells, lymphoid tissue inducer-like cells, B cells, natural killer cells, dendritic cells, intraepithelial lymphocytes, macrophages, natural killer T cells, and/or neutrophils. In some embodiments, the iIO comprises enterocytes, goblet cells, Paneth cells, and enteroendocrine cells. In some embodiments, the iIO comprises Villin+, Mucin2+, Lysozyme+, and/or Chromogranin A+ cells.

In some embodiments, upon exposure of the lumen of the iIO to an immune stimulating material, for example, an allergen, and/or a microbial lysate (e.g., of Escherichia coli) microfold cells (M cells) of the iIO express GP2 at the cell surface. In some embodiments, upon exposure of the lumen of the iIO to a lysate of Escherichia coli, plasma cells of the iIO produce IgA antibodies. In some embodiments, upon exposure of the lumen of the iIO to a lysate of Escherichia coli, M cells and B cells of the iIO are co-localized.

In some embodiments, the iIO comprises activated immune cells. In some embodiments the immune cells are activated by exposure of the lumen of the iIO to an immune stimulating material, for example, an allergen, and/or a microbial lysate. In some embodiments, the iIO with activated immune cells comprises GP2+ microfold (M) cells. In some embodiments, the GP2+ microfold (M) cells appear after a period of time post-exposure to the immune stimulating material (e.g., allergen, and/or microbial lysate). In some embodiments, the mucus of the iIO with activated immune cells comprises secreted IgA antibodies. In some embodiments, M cells and B cells colocalize within the iIO with activated immune cells.

### Compositions Comprising Human Intestinal Organoids With Human/humanized Immune Cells

In some embodiments, the IO of the iIO is an HIO. In some embodiments, the immune cells of the iIO comprise human and/or humanized immune cells. In some embodiments, the IO is an HIO, and the HIO comprises human immune cells and/or humanized immune cells in one or more layers of the HIO (hiHIO).

### Disease Model Organoid Compositions

Also disclosed herein are methods of making and compositions of disease model iIOs. In some embodiments, the iIO resembles at least one GI disease state. In some embodiments, the GI disease state is an allergy and/or infectious disease. In some embodiments, the GI disease state is induced by a genetic modification, for example a genetic modification of the cells from which the IO is derived and/or the immune cells. The genetic modification can be a naturally occurring mutation or it can be induced by known procedures. In some embodiments the IO are prepared from tissue from subjects suffering from a GI disease, for example a genetic disease. In some embodiments, the GI disease state is induced by exposure to a compound, an allergen, and/or a pathogen, either before the formation of the IO (e.g., exposing the iPSCs or DE cells), or after the IO and/or iIO is formed (e.g., exposure of the lumen of the IO or iIO to the compound, an allergen, and/or a pathogen).

### Methods of Use

In some embodiments, the iIO is utilized to develop a mucosal vaccine. Also disclosed herein are methods of treatment comprising transplanting an iIO disclosed into an organism, for example, a subject suffering from a GI disease state. Also disclosed herein are methods of screening a compound for activity, the method comprising contacting an iIO, or population of iIOs, disclosed herein (e.g., a normal and/or a GI disease state iIO or population of iIOs) with the compound, for example a potential or known therapeutic agent, and measuring a response of the iIO to the compound. In some embodiments the screening is to determine the efficacy and/or toxicity of a compound, for example in treating a GI disease state. In some embodiments, the iIO is a model for a GI disease, and assessing the effects of the candidate compound or composition on the iIO organoid comprises assessing the effects of the candidate compound or composition on the GI disease. In some embodiments, the iIO organoid has been produced from cells derived from a subject. In some embodiments, the cells derived from the subject are induced pluripotent stem cells. In some embodiments, the subject has or is disposed to develop a GI disease.

### Embodiments of an in Vivo Model of Human Io Having Functional Human Immune Tissues

In an embodiment disclosed herein, a next generation in vivo model of HIOs with functional human immune tissue is reported (see Examples for additional details). Using this model, it is demonstrated that immune cells temporally infiltrate and populate the HIO in the mucosa comparable to the immune landscape in developing human gut. Interestingly, mass cytometry data and immunostaining demonstrated the formation of GALT-like structures during HIO development and their formation correlated temporally and spatially with human intestinal immune tissue development. Because M cells are present in the epithelium overlaying the GALT, their presence was confirmed and their function was validated by exposing the HIO lumen to microbial components.

The gut immune system is distributed throughout the epithelium, the lamina propria and in a network of lymphoid structures called the GALT ranging from a complex and well-organized structure. Intestinal immune tissue is comprised of diverse immune cell types to protect the mucosal barrier against any invaders. Recent publications have demonstrated that the human fetal gut already expresses a diverse and functional immunity even without being exposed to antigens. In the mass cytometry data, the analysis indicated that the immune cell composition was diverse and increased in cell number as the transplanted HIO developed. The results correlate with recent published mass cytometry data and transcriptomics in which an influx in immune cell number as well as cell subsets, in particular CD4+ T cells, was found during human fetal intestinal development. During HIO development, an increase in CD4+ T and B cell frequencies was found by mass cytometry and confirmed by histology that these cell types together formed aggregates later developing into lymphoid follicles. These features were not identified in the intestine of humanized mice.

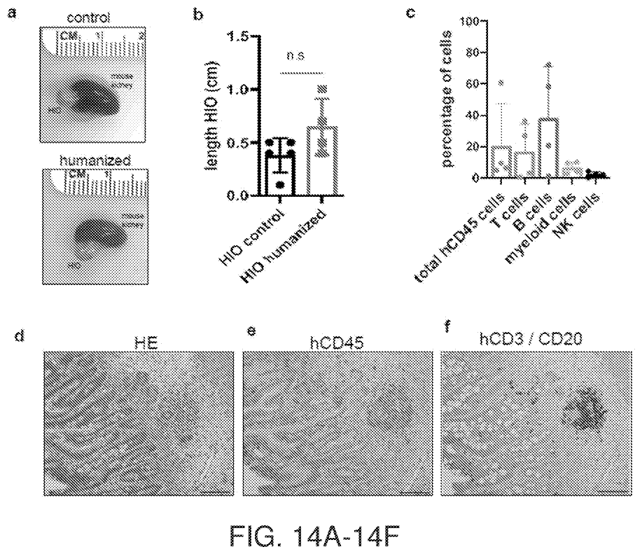

In embodiments disclosed herein, the timing of humanization and transplantation did not affect the timing of GALT structure formation. In a preliminary study delaying the timing of transplant until the immune system was fully reconstituted, histology and flow cytometry studies indicated that immune cellular aggregates composed by T and B cells were able to develop similarly to our reported data when HIO were transplanted early (FIG. 14). These observations first indicate that immune precursors are present even after the immune system was reconstituted. It appears the developing HIO and cells seeding the lamina propria influence and frame the formation of immune aggregates by expressing specific signals to attract and activate immune precursors as well as serve as a foundation for subsequent lymphoid tissue development.

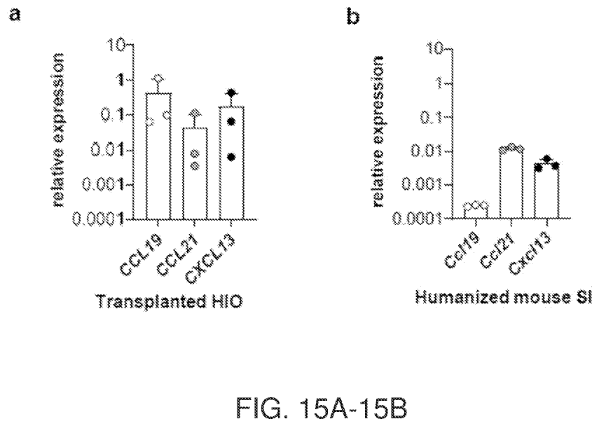

Recently, transcriptomic analysis on human fetal intestine have identified subsets of fibroblasts in GALT formation that correspond to lymphoid tissue organizer (LTo) cells. It has been found that the expression of these cells increased with fetal development and interact with diverse immune LTi cells via the expression of CCL19, CCL21 and CXCL13. In this model, preliminary data indicated that CCL19, CCL21 and CXCL13 genes were expressed in HIOs at 16 weeks post-transplantation whereas their mouse gene orthologs were decreased by at least 10-fold (FIG. 15). These results indicate that, in contrast to the humanized mouse small intestine, the HIOs express unique microenvironment signals that support the formation of GALT-like structures. In line with this, this model could be used to dissect the mechanism of interaction between stromal LTo with LTi cells and identify key pathways in the development of GALT in human.

GALT are sites of adaptive immune responses and influence the epithelial cells overlaying follicles to express M cells that transport luminal antigens to activate immune cells. GP2 is a glycoprotein expressed on the apical side of M cells and translocate luminal antigens to the immune cells in the lamina propria. Even though transplanted HIOs expressed GP2 gene, the protein was not detected by immunohistochemistry likely due to the lack of microbial antigens in the lumen similarly to a fetal intestine. Using the current model, it was demonstrated that exposing the HIO lumen to E. coli lysate induced the expression of M cells characterized by GP2 expression as well as activation of an immune response indicated by the presence of IgA antibodies. The release of IgA antibodies in the lumen occurs by transcytosis across epithelial cells. After being produced by plasma cells in the lamina propria, IgA antibodies bind to polymeric immunoglobulin receptors (pIgR) expressed on the basal side of epithelial cells and by transcytosis, epithelial cells then secrete IgA antibodies in the intestinal lumen. Because the presence of IgA antibodies was detected in the mucus, the findings show that epithelial cells in HIOs express pIgR and transport antibodies to the lumen.

### EXAMPLES

### Example 1

### Integrating Immune Cells in Human Intestinal Organoids (HIOs)

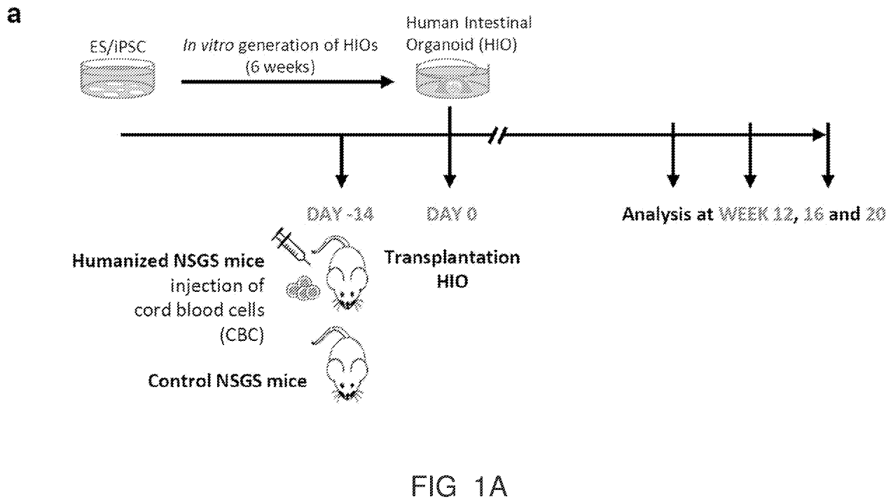

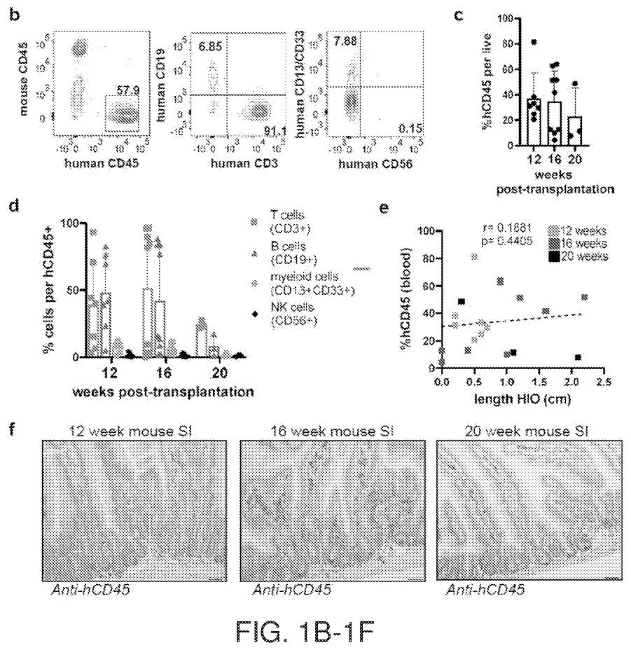

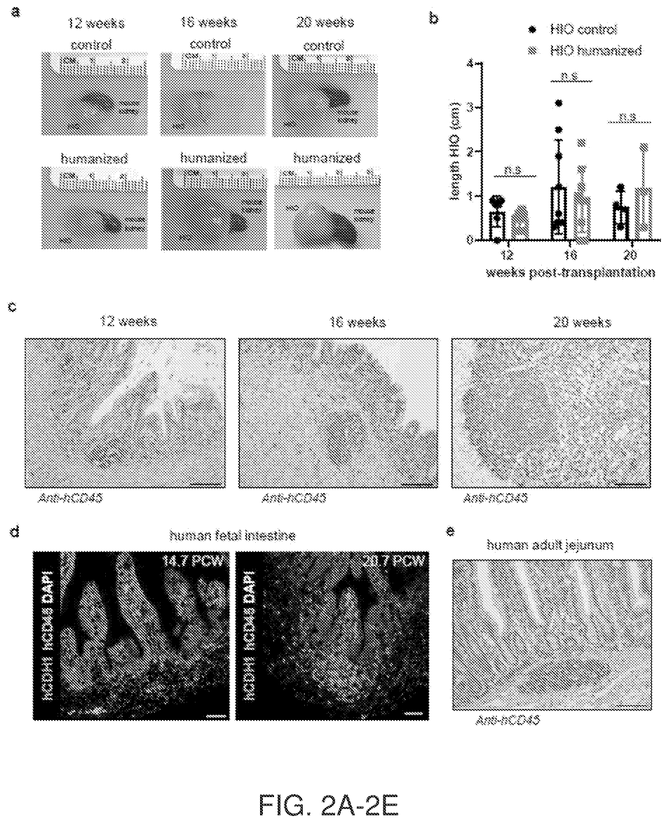

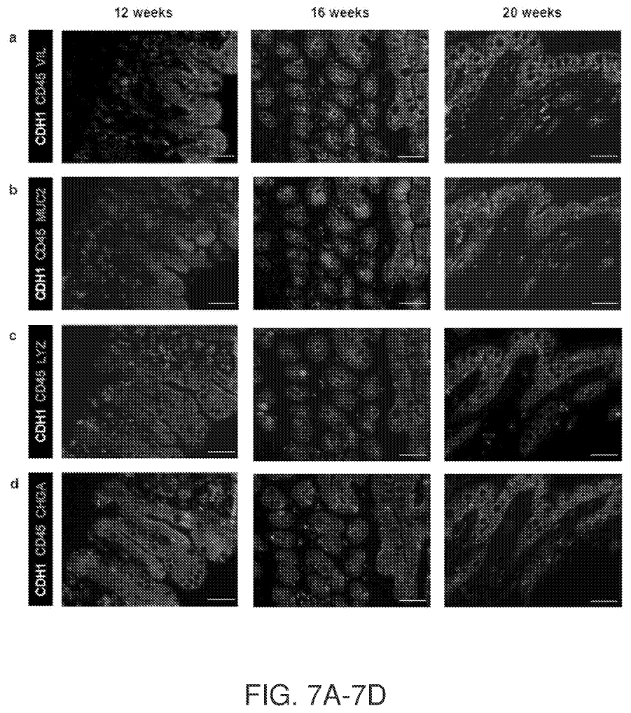

To bioengineer an HIO with immune cells, PSC-derived HIOs were transplanted into humanized NSGS mice (see workflow in FIG. 1A). Once the human immune system in mice was fully reconstituted and repopulated peripheral organs, HIOs were harvested and analyzed at 12, 16 and 20 weeks post transplants. The presence of human immune cells in the blood of humanized mice was confirmed at each time point (FIG. 1B-D), and HIOs successfully engrafted and grew at similar size compared to non-humanized control mice (FIG. 2A-B). Compared to 12 weeks, HIOs transplanted for a period of 16 and 20 weeks were more heterogeneous in size and this phenomenon may be due to an accumulation of mucus within the HIO lumen which cannot be drained out. Regardless, the effect is independent of the presence of immune cells as the same heterogeneity in size was observed at 16 and 20 weeks in control mice. In addition, there was no correlation between the size of HIO and the percentage of human immune cells in the peripheral blood of humanized mice (FIG. 1E). By immunohistochemistry (IHC) staining, it was demonstrated that human CD45+ cells had migrated to the mucosal layer and populated the lamina propria as well as the epithelium and formed cellular aggregates comparably to a human fetal and adult gut immune landscape (FIG. 2C-E). Notably, this did not occur within the humanized mouse small intestine (FIG. 1F). HIOs engrafted and differentiated in humanized mice, expressing all major intestinal cell lineages (FIG. 7).

### Example 2

### Mass Cytometry Analysis Reveals a GALT-like Immune Signature Profile in HIO

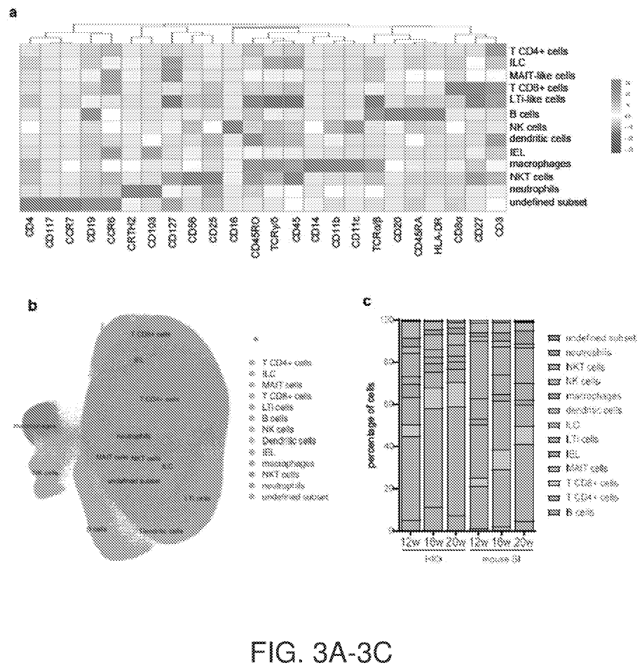

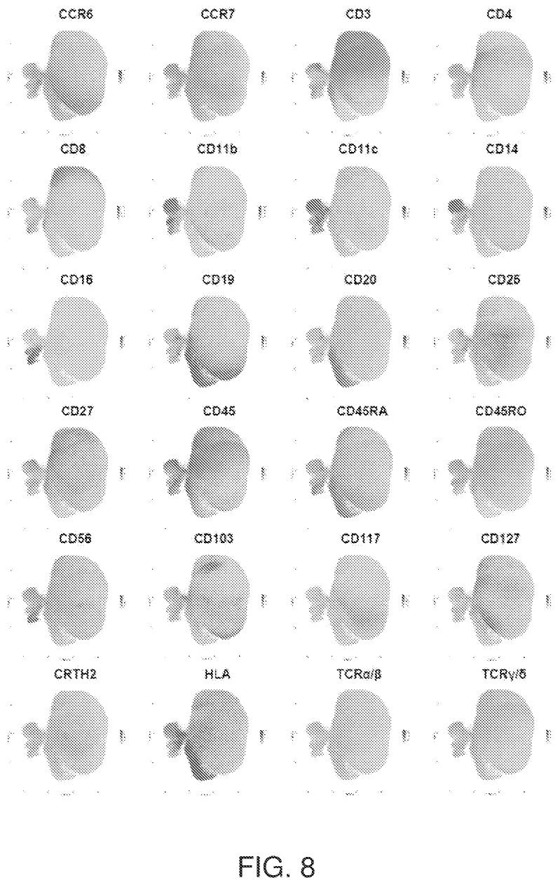

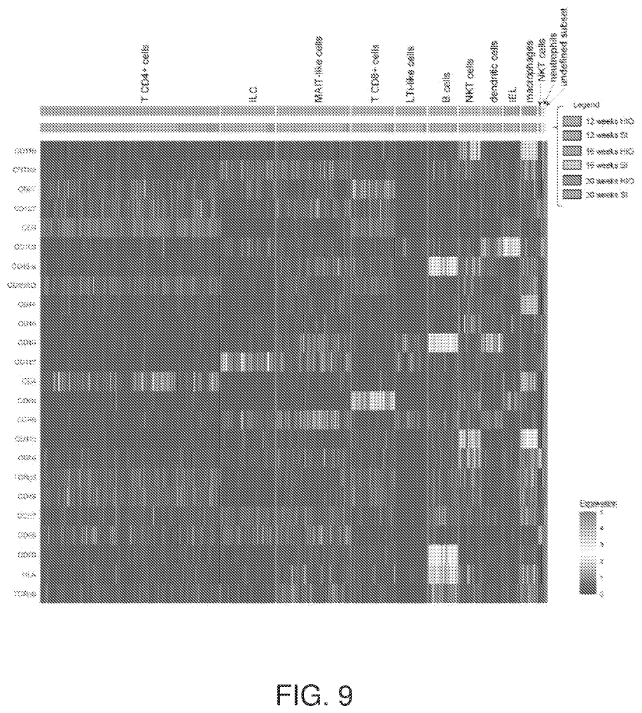

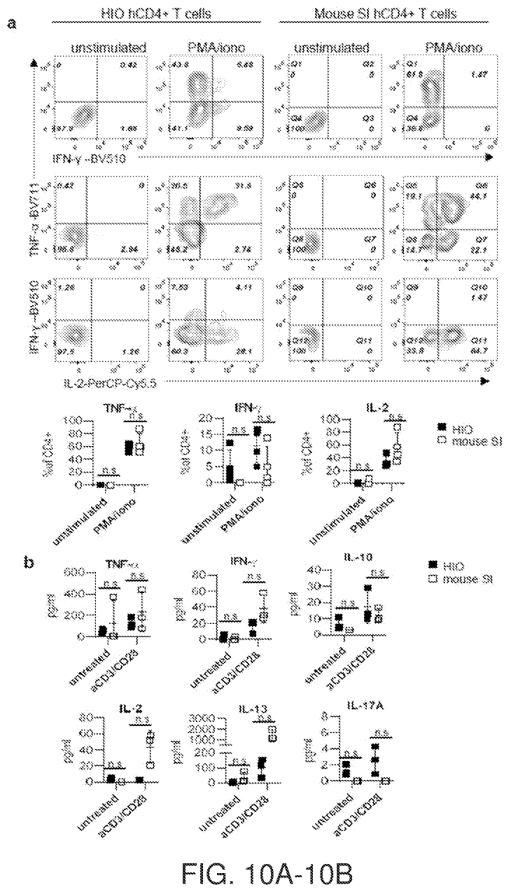

By mass cytometry, the immune signature in transplanted HIO was determined, and was compared to the humanized mouse small intestine (SI) as an internal control. Using unsupervised clustering analysis, human CD45+ immune cells were classified and organized into 13 clusters as visualized in a heatmap and UMAP (Uniform Manifold Approximation and Projection) graph (FIG. 3A-B). Based on the combination and level of expression of markers on the heatmap and expression pattern on individual UMAP graph (FIGS. 3A, 8, and 9), 12 immune cell types were defined corresponding to the major immune cell lineage found in the intestine, for instance T, B cells and innate lymphoid cells (ILC), as well as tissue-specific immune cells such as intraepithelial lymphocytes (IEL), mucosal-associated invariant T (MAIT)-like cells and lymphoid tissue inducer (LTi)-like cells (FIG. 3A-B). To compare the immune profile between HIO and humanized mouse SI post HIO transplantation, the frequencies of each immune cluster in each group were determined at each time point (FIG. 3C). The results revealed a comparable immune cellular composition between the groups except a differential expression of T cells, particularly CD4+ T cells, as well as B cells. B cells and CD4+ T cells were present at higher levels in HIOs compared to humanized mouse SI and their frequency increased from 12 to 16 weeks. However, further expression was not seen in the immune profile at 20 weeks post HIO transplantation compared to the profile at 16 weeks. There was no inflammation associated with the increase in CD4+ T cells, as demonstrated by a similar cytokine profile in cells isolated from HIOs or humanized mouse SI (FIG. 10A-B). Interestingly, in the small intestine, B cells are mostly found in gut-associated lymphoid tissue (GALT) structures such as lymphoid follicle or Peyer's patch that also contained CD4+ T cells. Because the humanized mice lack IL-2Rγ, they do not express intestinal lymphoid follicles and Peyer's patches, resulting in less T and B cells present in the intestine which correlate with the mass cytometry results. Taken together, the findings show that the augmentation of B and T cell frequencies observed in HIOs could come from GALT structures.

### Example 3

### Immune Aggregates in the Developing HIO are Lymphoid Follicle-like Structures

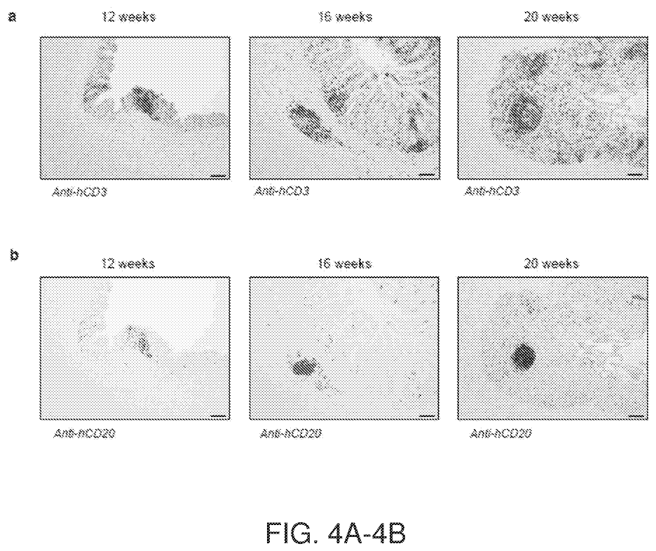

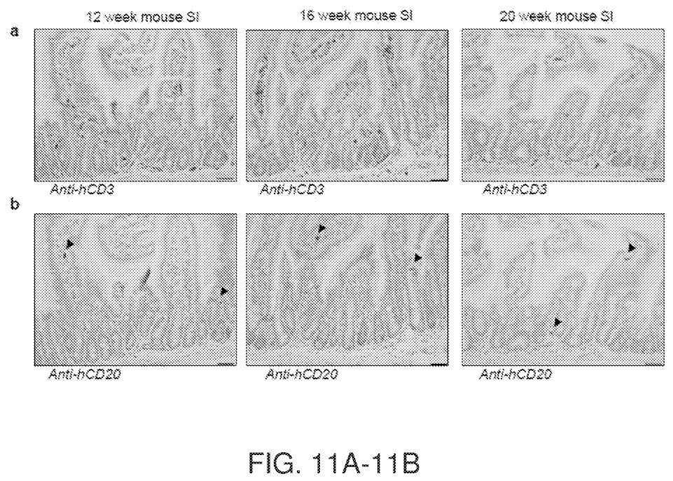

Lymphoid follicles are well-organized structures formed by aggregation of B cells surrounded by T cells. IHC staining with anti-human CD3 and CD20 antibodies revealed that aggregates found in HIOs contained T and B cells, respectively, at all time points (FIG. 4). Surprisingly, in contrast to 12 week-old transplanted HIOs, a distinct cellular zonation of T and B cell populations appeared in 16 as well as 20 week-old HIOs resembling lymphoid follicle-like structure (FIG. 4). However, aggregates of T and B cells were not observed in the humanized mouse small intestine at any time point (FIG. 11). This result shows that HIOs promote and influence the formation and maturation of lymphoid follicular structures over the course of its development.

### Example 4

### Temporal and Spatial Development of Lymphoid-like Structures in Transplanted HIOs Correlate to Human Lymphoid Follicle Development

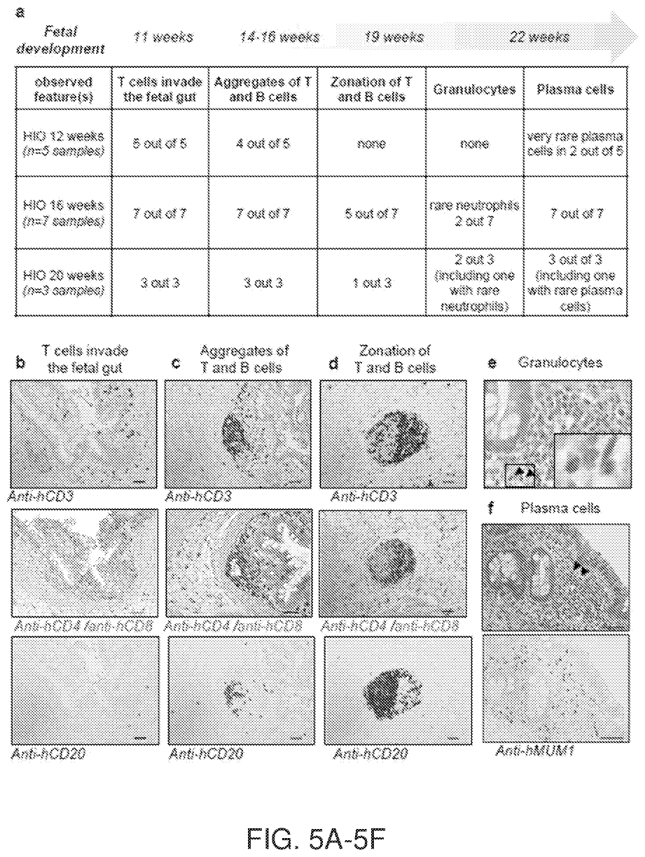

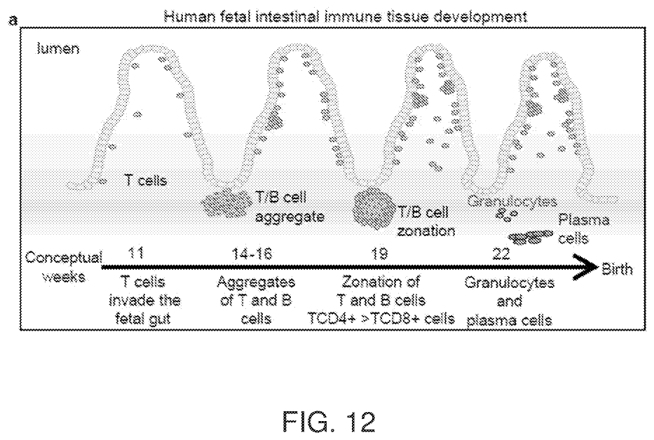

Because the developing HIO is fetal in nature and the lumen lacks exposure to antigens and microbiota, immune aggregates developing in HIO were compared to the developing human fetal gut. It has been reported that lymphoid follicles develop in the human fetal gut starting at the second trimester of gestation. In FIG. 12, previous findings on the developing human fetal gut immune tissue were summarized and used as a reference to compare with the features observed in transplanted HIOs. The 12 and 16 week time points were analyzed since the mass cytometry and histology results indicated a similar immune profile between 16 and 20 weeks. At 12 weeks post HIO transplant, the presence of T cells (mostly CD4+ cells) with few scattered CD20+ B cells were found in the lamina propria as well as the epithelium pointing out similarity with cellular composition of a fetal intestine at 11 post conceptual weeks (PCW) (FIG. 5A-B). Additionally, in some areas, the co-localization of T and B cells that resemble the aggregates described in the fetal intestine at 14-16 PCW was observed (FIGS. 5A, 5C). Interestingly, the cellular zonation of T and B cells observed in the fetal intestine at 19 PCW, was found in 16-week HIOs where T-cell zone are represented with high proportion of CD4+ T cells and low CD8+ T cells (FIGS. 5A, 5D). As described in human fetal intestine, the presence of plasma cells and few neutrophils was observed in 16-week HIOs demonstrating a more mature and complex cellular composition over the course of the HIO differentiation (FIGS. 5A, 5E). Importantly, these data confirm that the HIO environment expresses unique signals to influence the maturation of the immune system as well as the formation of a lymphoid tissue.

### Example 5

### M Cells Are Induced in Transplanted HIO After Microbial Exposure

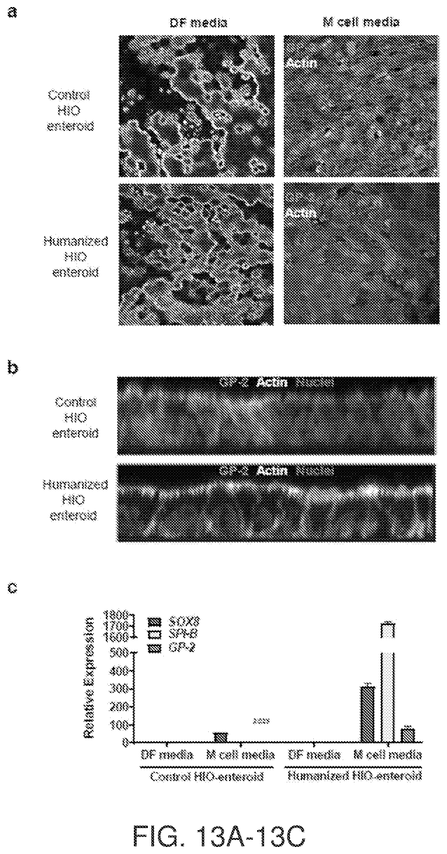

Cross-talk between lymphocytes and epithelial cells are known to have an influence on M cell differentiation. M cells are expressed in the epithelium overlying follicles and play a key role in immune responses by transporting antigens from the lumen in the lamina propria. By histology, GP2, a marker for M cells, was not detected at baseline in HIOs post-transplantation. However, in enteroids derived from HIO epithelium, GP2 was induced in vitro and expressed at high level in enteroids derived from HIOs transplanted in humanized mice compare to control mice (FIG. 13).

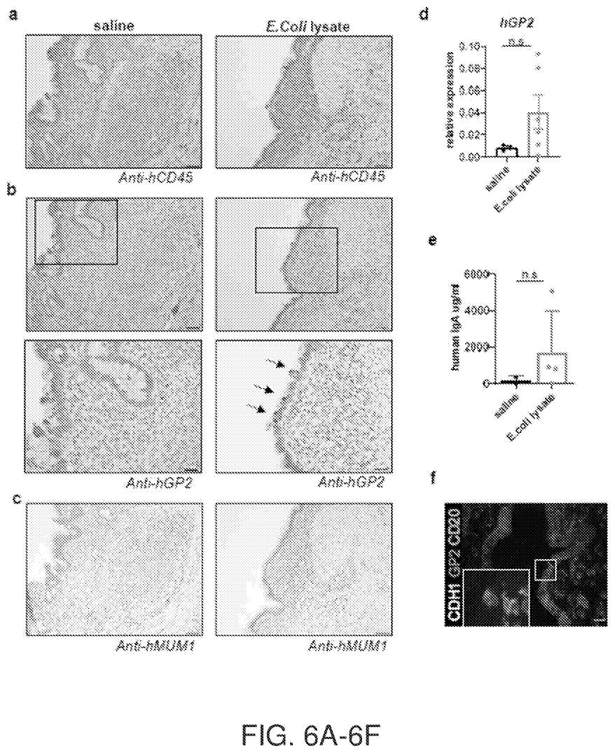

GP2 is a transcytotic receptor and binds FimH an adhesin molecule expressed by Escherichia coli (E. coli). It was reported that the number of M cells increased after bacterial challenge. Therefore, the induction of M cells after bacterial exposure in HIO 16 weeks post-transplantation was determined since their immune profile is more mature than at 12 weeks. The results demonstrated that 72 hours after administration of E. coli lysate in HIO lumen, GP2 was detected on the cell surface of epithelial cells overlying immune cells, indicating the presence of M cells (FIGS. 6A, 6B). In contrast, transplanted HIOs injected with saline had minimal background expression probably induced by antigens present in the environment at the time of injection. In line with the histology results, the induction of GP2 expression in transplanted HIOs after E. coli exposure compared to saline group was confirmed by qPCR (FIG. 6D). M cell-mediated translocation of luminal antigens initiates the production of IgA antibodies. To determine whether M cells were functional and able to translocate luminal antigens to activate immune cells, the level of IgA secreted in the mucus of transplanted HIO was measured in response to E. coli lysate injection. Even though, plasma cells are present in both groups, unlike in saline-treated group, IgA antibodies were present at high level in the mucus of HIOs exposed to E. coli lysate (FIGS. 6C, 6E). This result indicates that M cells are functional in translocating antigens to activate immune cells which subsequently respond to the microbial exposure by activating plasma cells to produce IgA antibodies. It was then confirmed in E. coli-treated group a colocalization of M cell and B cell in by immunofluorescence, indicating a crosstalk between epithelial and immune cells (FIG. 6F). Altogether, the induction of M cells expression in HIO offers unique opportunities to model human GI diseases such as allergy or infectious diseases where the presence of diet or infectious components are translocated to the lamina propria to activate immune cells.

### Example 6

### Materials and Methods

### Animals.

Immunodeficient NOD/Scid 112 rg null Tg (hIL3, hGM-CSF and hSCF) (NSGS; The Jackson laboratory #013062) mice were bred and housed in the animal facility at the Cincinnati Children's Hospital Medical Center (CCHMC). All experiments were performed with the approval of the Institutional Animal Care and Use Committee of CCHMC (IACUC protocol #2018-0092).

### Human Intestinal Tissue.

Normal, de-identified developing human fetal tissues from elective abortions were obtained from the University of Washington, Laboratory of Developmental Biology, and all work was approved by the University of Washington and the University of Michigan Institutional Review Board (IRB).

Normal adult human jejunum were obtained from patients undergoing bariatric procedures between the ages of 16 and 25 years old. Informed consent or assent was obtained from all patients and/or parent/legal guardians as appropriate. Human tissue collection was performed with the prior approval of Cincinnati Children's Hospital Medical Center's (CCHMC) IRB.

### Human Intestinal Organoids (HIO)

Human intestinal organoids were generated and maintained as previously described.

Briefly, human HI embryonic stem(ES) cells (WA-01; WiCell) (passage number 40 to 55) obtained from the Pluripotent Stem Cell Facility in the institute, were grown in feeder-free conditions in mTESR1 media (Stem Cell Technologies). For induction of definitive endoderm (DE), cells were split with Accutase (Invitrogen) and plated at a density between 70,000 and 100,000 cells per well in a Matrigel-coated 24-well plate. Once the cells reached 80 to 95% confluency, they were treated with 100 ng/ml of Activin A for 3 days as previously described. DE was then treated for 4 days with hindgut induction medium containing 500 ng/ml FGF4 (R&D) and 3 μM Chiron 99021 (Tocris) to induce formation of mid-hindgut spheroids. Spheroids were then plated in Matrigel (Corning) and maintained in intestinal growth medium supplemented with 100 ng/ml EGF (R&D) to generate human intestinal organoids (HIOs). Media was changed twice weekly and HIOs were replated in fresh Matrigel at 14 days.

### Human Umbilical Cord Blood Cell Engraftment.

The Translational Trials Development Support Laboratory of CCHMC collected and distributed the Umbilical Cord Blood (UCB) units according to an approved IRB protocol (protocol #02-3-4×). Mice were humanized as described previously. Briefly, whole cord blood was subjected to hetastarch induced aggregation of RBCs. Cord blood cells (CBCs) were isolated, washed, and viably frozen for future use. Thawed CBCs were resuspended in IMDM media with 3% FBS and antibiotics and diluted to 23.3-28.3×10⁶ CBCs/mL. OKT3 antibody was spiked into the cell solution at a concentration of 1 μg per 1 million cells to prevent GVHD. 6-8 weeks old immunodeficient NSGS mice were conditioned by receiving a dose of busulfan (30 mg/kg by ip injection) 24 hours prior to intravenous injection of 7-8.5×10⁶ CBCs in 300 μL. Busulfan is a chemotherapy drug that partially depletes cells from the mouse bone marrow and allows the human hematopoietic stem cells to engraft. As a preventative measure against possible systemic infections, mice were fed with doxycycline chow (0.0625%, Purina) 2 weeks prior and after human cell engraftment.

### Transplantation of HIOs.

As previously described, a single HIO, matured in vitro for 28 days, was removed from Matrigel and then transplanted under the kidney capsule. Briefly, the mice were anesthetized with 2% inhaled isoflurane (Butler Schein) and 2.5-3 L/min oxygen. The left side of the mouse was then prepped in sterile fashion with isopropyl alcohol and providine-iodine. A small left-posterior subcostal incision was made to expose the kidney. A subcapsular pocket was created in the kidney capsule and the HIO was then placed into the pocket. The kidney was then returned to the peritoneal cavity and the mice were given an intraperitoneal flush with 2-3 mL of piperacillin/tazobactam (100 mg/kg; Pfizer Inc.) to help prevent bacterial infection. The skin was closed in a double layer. For pain control, mice were then given a subcutaneous injection with Buprenorphine (0.05 mg/kg; Midwest Veterinary Supply) or Carprofen (4 mg/kg Midwest Veterinary Supply) and were monitored for the next 48 hours following surgery. Additional injections of pain medication were given if needed. At 12 and 16 weeks following engraftment, the mice were then euthanized and the tissues were harvested and analyzed.

Escherichia Coli (E. coli) lysate preparation and injection. Grown in LB media overnight, E. coli suspension was then washed three times with PBS and centrifuge at 1000×g. Bacteria were resuspended in saline solution at 10⁷ CFU/mL and lyzed by freeze-thaw cycles repeated 4 times.

16 weeks post HIO transplantation, the mice were anesthetized and small left-posterior subcostal incision was made to expose the transplanted HIO. 50 to 100 μL of E. coli lysate were injected with a 0.5 cc insulin syringe in the HIO lumen.

The HIO was then returned to the peritoneal cavity the skin was closed in a double layer. For pain control, mice were then given a subcutaneous injection with Carprofen (4 mg/kg Midwest Veterinary Supply) and were monitored for the next 48 hours following surgery. Additional injections of pain medication were given if needed. At 72 hours post injection, the mice were then euthanized and the tissues were harvested and analyzed.

### Flow Cytometry.

To confirm the expression of human immune cells from CD34+ engraftment in mice, retro-orbital bleeding was performed 8 to 10 weeks after the engraftment and a day prior each tissue harvest. ˜50 μL of whole blood was collected in a BD Microtainer Dipotassium/EDTA coated tube and then lysed in 5 mL of Red Blood Cell (RBC) lysis buffer (155 mM NH4Cl, 12 mM NaHCO3 and 0.1 mM EDTA pH8.0; diluted in dH2O), for 5 min at room temperature. Isovolume of PBS was added and tubes were centrifuged at 300×g for 5 min. The pellet was washed in PBS, centrifuged, and resuspended in FACS buffer (0.5% BSA, 2 mM EDTA in PBS 1×). Cells were stained for 30 min on ice with the following combination of antibodies FITC-conjugated anti-human CD45, PE-Cy5-conjugated anti-mouse CD45, BV650-conjugated anti-human CD19, PE-Cy7-conjugated anti-human CD3, BV421-conjugated anti-human CD56, PE-conjugated anti-human CD13, PE-conjugated anti-human CD33 and Zombie NIR fixable viability kit in order to exclude dead cells. All antibodies were used at 1:200 dilution except Zombie NIR which was used at a 1:2000 dilution. Samples were washed twice and resuspended in FACS buffer. The samples were then recorded on a LSR Fortessa instrument (BD Biosciences) and the data was analyzed with FlowJo software (TreeStar, Inc).

### HIO and Small Intestine Single-Cell Preparation.

One third of the HIO was used for cell dissociation. Cell dissociation protocol for the HIOs has been modified from Weigmann's protocol for mouse colon cell dissociation. Briefly, HIOs were cut in small pieces and incubated under slow rotation for 20 min at 37° C. in 5 mL of predigestion solution containing EDTA and DTT in HBSS. The epithelial cell suspension was filtered through a 100 um cell strainer, washed with cold PBS and kept on ice until pooled with the lamina propria-isolated cells. The remaining pieces of HIO were minced and placed in a new tube with 5 mL of digestion solution containing collagenase D (Roche), DNAse I (Roche) and dispase II (Roche) in PBS and incubated for 15 min at 37° C. under slow agitation. Lamina propria cell suspension was passed through a 70 μm cell strainer, washed with cold PBS and re-incubated in 5 mL of digestion solution for 15 min at 37° C. under slow agitation. This step was repeated one more time. Epithelial and lamina propria cells were combined in one tube and spun down at 450×g for 5 min.

The method to isolate mononuclear cells from the humanized mouse gut was adapted from the protocol reported by Lee et al. Briefly, 10 to 12 cm of proximal small intestine was harvested, longitudinally cut open and washed in HBSS to remove any debris. The small intestine was incubated in HBSS containing 5 mM EDTA on ice for 5 min and vortex at medium intensity, for a total of 4 incubations. The epithelium was then collected in a separate tube. After digestion of the tissue at 37° C. for 30 min with DNAse I (Roche) and Collagenase A (Roche), the homogenate was filtered and centrifuged at 450×g for 5 min.

Both, HIO and small intestine cell suspensions were individually resuspended in 44% Percoll (GE), loaded on a 67% Percoll layer and centrifuged at 650×g without brake for 30 min at room temperature. The cell layer at the interface of the 2 gradients was collected, washed, counted and used for in vitro assays or stained for immune markers and analyzed by mass cytometry.

### Mass Cytometry Staining and Data Analysis.

### Mass Cytometry Staining.

Antibodies used in this panel were purchased from Fluidigm except anti-Human CD45RO antibody was purchased from BioLegend and was labelled with Maxpar X8 Antibody Labelling Kit (Fluidigm) according to the manufacturer's instructions. All the reagents used in the following protocol were purchased from Fluidigm and all incubations were done at room temperature. Samples were first stained for 5 min with Cell-ID Cisplatin at a final concentration of 5 μM in Maxpar PBS and then washed in 5 volume of Maxpar Cell Staining Buffer. Before being stained for cell surface markers, cells were fixed in Fix I Buffer for 10 min, washed twice with Maxpar Barcode Perm Buffer and barcoded with Cell-ID 20-Plex Pd Barcoding Kit for 30 min. Samples were pooled and then stained for 30 min with a cocktail of 25 antibodies in a final volume of 100 μL of Maxpar Cell Staining Buffer (antibody dilution 1:200). After cell surface staining, samples were washed, fixed in 1.6% paraformaldehyde (PFA) (EMS) for 10 min and finally, incubated in 1 mL of intercalator solution at a final concentration of 125 nM. The samples were kept cold and shipped in intercalator solution to the Cytometry Research Core Facility at the University of Rochester NY. The samples were washed and resuspended in Maxpar water before being acquired using a CyTOF/mass cytometer Helios instrument (Fluidigm). Data were then debarcoded using Fluidigm debarcoder software (available online).

### Bioinformatics Analysis.

CyTOF datasets from transplanted HIO- and humanized mouse small intestine (SI)-isolated immune cells were generated using Mass cytometry (Fluidigm) variation at three time points 12 weeks, 16 weeks and 20 weeks. Datasets at aforementioned time points were generated in two batches for each tissue at 12 and 16 weeks post transplantation with a total of n=7 samples and n=10 samples, respectively, and one batch for each tissue at 20 weeks post transplantation. Combining two batches, ˜1.1 million cells were analyzed accounting to 63,076 cells in 12 weeks HIO, 350,132 cells in 16 weeks HIO, 148,274 cells in 20 weeks HIO, 137,887 cells in 12 weeks SI, 368,893 cells in 16 weeks SI, and 71,569 cells in 20 weeks SI.

Raw .fcs files in each sample was read and analyzed using read.flowSet function from flowCore R (v3.6.3) package. Fcs files stored in flow. set objects were than normalized using inverse hyperbolic sine (Arcsinh) transformation (https://support.cytobank.org/hc/en-us/articles/206148057-About-the-Arcsinh-transform) using cofactor value of 5. Normalized reads from the fcs files were then used to create a Seurat object using CreateSeuratObject function from Seurat (v3.0.2) a single cell analysis package in R.

For each Seurat object, following steps were carried out: variable features set to surface markers, scaling, dimension reduction and clustering. Dimension reduction was applied in both linear [principal components (PCs)] and non-linear [uniform manifold approximation projection (UMAP)] approach to obtain respective components. Clustering was performed using K-nearest neighbor (KNN) graph of cells based on marker abundance similarity. This KNN graph is finally partitioned into clusters based on highly interconnected colonies of cells.

In the end, all the processed Seurat objects for each sample from two batches are integrated together using Seurat's reciprocal principal component analysis (RPCA) as the number of cells were large in number (˜1.1 million). Briefly, each dataset is projected into principal component space (PCS) of other datasets to learn the anchors (cell pairs) based on mutual nearest neighbor graph requisite.

Dimension reduction plots were generated using DimPlot function and heatmaps were generated using the DoHeatmap function in Seurat.

Example datasets and analysis code has been deposited to GitHub: https://github.com/praneet1988/Analyze_CyTOF_Using_Seurat.

Measurement of Cytokines from HIO and Mouse Small Intestine-Derived Immune Cells.

### Detection of Cytokines by Flow Cytometry.

Immune cells isolated from HIO or mouse small intestine were stimulated for 4 hours with 1:500 of Cell Activation Cocktail (BioLegend) containing, according to the manufacturer, optimized concentration of phorbol 12-myristate-13-acetate (PMA) and ionomycin. After an hour of stimulation, cells were then incubated with 1:1,000 of Brefeldin A Solution (Biolegend) to block the secretion of cytokines. Finally, the cells were stained with anti-human CD3 and anti-human CD4 antibodies (Biolegend), fixed with Cytofix (BD Biosciences) and then permeabilized with CytoPerm (BD Biosciences) overnight at 4° C. The following day anti-IFNγ, anti-TNFα and anti-IL-2 antibodies (BD Biosciences and Biolegend) were added to the cells for at least 1 hour at room temperature. Samples were washed twice and resuspended in FACS buffer. The samples were then recorded on an Aurora instrument (Cytek) and the data was analyzed with FlowJo software (TreeStar, Inc).

### Detection of Cytokines by Milliplex Assay.

Indicated cytokines were measured in supernatants from immune cells (cell density 10⁶/mL) isolated from HIO or mouse small intestine stimulated for 3 days with a cocktail of anti-human CD3/anti-human CD28 antibodies (STEMCELL Technologies). The presence of cytokines in supernatants were measured using Milliplex kits (Millipore), following the manufacturer's instructions.

In vitro HIO-Derived Enteroid Culture (M Cell Induction).

### HIO-Derived Enteroid Preparation and in Vitro Expansion.

At the time of harvest, a section of transplanted HIOs was used to isolate the crypts following our protocol for human intestinal tissue. Briefly, the mucosal layer from portions of transplanted HIOs was dissected under a microscope and scraped to remove the villi and debris. The mucosa was then incubated with freshly prepared 2 mM EDTA solution and gently shake for 30 min. After several washes with ice-cold chelation buffer, the intestinal crypts were collected by gently scraping the mucosa with curved forceps and filtered twice through a 150 μm nylon mesh to remove any debris. Due to limited amount of tissues, the crypts collected from each group were pooled. The crypts were then washed in ice-cold chelation buffer and 50 μL of crypts, resuspended in Matrigel (Corning), were added per well in a 24-well plate. After polymerization of the Matrigel, 500 μL of human IntestiCult Organoid Growth medium (STEMCELL Technologies) were added to each well. The crypts were cultured and expanded for 10 to 14 days before being frozen down for a later use.

### M Cell Induction in Vitro.

Enteroids were plated on Transwells as described previously. Briefly, after being washed from Matrigel, enteroids were fragmented and plated on human collagen IV-precoated 24-well plate Transwells (0.4 μum pore size) and incubated in IntestiCult Organoid Growth medium (STEMCELL Technology) at 37° C. until monolayer confluence is reached. To induce M cell differentiation, enteroid monolayers were cultured in differentiation (DF) medium supplemented with 50 ng/mL TNF-α and 100 ng/ml RANK-L for a period of 5 days, as previously described.

### Histology Staining.

### Immunohistochemistry Staining.

HIO and mouse small intestine tissues were fixed overnight in 4% paraformaldehyde (PFA) at 4° C., paraffin embedded and sectioned at 5 μm. For human CD3, CD20, CD4, CD8 and MUMI staining, the slides were prepared by the Pathology Core at CCHMC using Automation VENTANA BenchMark instruments. For human CD45 staining, sections were prepared as previously described. Briefly, sections were deparaffinized, subjected to antigen retrieval in Dako solution pH6 (citric acid), permeabilized in 0.5% TritonX in PBS and blocked for 1 hour at room temperature in PBS/1% BSA supplemented with serum and then incubated overnight at 4° C. with primary antibody diluted in PBS/1% BSA. Next day, slides were washed and incubated overnight at 4° C. with biotinylated secondary antibody diluted in PBS/1% BSA. Signals were amplified with RTU Vectastain ABC reagent (Vectorlab) and precipitated using DAB kit solution (Vectorlab) and finally counter stained with Mayer's hematoxylin solution (Dako). Images were captured on a Nikon Eclipse Ti and analyzed using Nikon Elements Imaging Software (Nikon).

### Immunofluorescence Staining on Human Fetal Intestinal Tissue.

Immunofluorescence staining was conducted as previously described. Briefly, human fetal intestinal tissue (˜0.5 cm fragments) were fixed in 10% neutral buffered formalin (NBF) for 24 hours at room temperature, then paraffin embedded and sectioned (5 μum thickness). Paraffin sections were first deparaffinized in Histo-Clear II (National Diagnostics) and re-hydrated. Antigen retrieval was performed by steaming slides in a sodium citrate buffer for 20 min. Slides underwent a blocking step using 5% normal donkey serum (diluted in PBS +0.5% Tween20) for 1 hour at room temperature. Human CDHI and CD45 primary antibodies were diluted 1:500 in blocking solution and slides were incubated with antibodies overnight at 4° C. The following day, slides were washed and incubated with secondary antibodies (1:500) diluted in a blocking buffer for 1 hour at room temperature together with DAPI staining (1 μg/mL). Slides were washed and mounted using Prolong Gold (Thermo Fisher). Imaging was done using a Nikon A1 confocal at the University of Michigan Medical School and images were assembled using Photoshop CC. Images were adjusted in Photoshop to optimize for visualization. For all images, any post-image processing (i.e. pseudocoloring, brightness, contrast, LUTs) was performed equally on entire images from a single experiment.

Immunofluorescence confocal imaging on HIO-derived enteroids. HIO-derived enteroid monolayers were fixed in aqueous 4% paraformaldehyde (PFA); (Electron Microscopy Sciences) for at least 30 min at room temperature, as previously described. Briefly, fixed monolayers were washed with PBS followed by simultaneous permeabilization and blocking in a solution of 15% FBS, 2% BSA, and 0.1% saponin (Sigma-Aldrich, USA) in PBS for 30 min at room temperature. Cells were rinsed with PBS and incubated overnight at 4° C. with primary mouse monoclonal antibody to human GP-2 diluted 1:100 in PBS containing 15% FBS and 2% BSA. Stained cells were then washed 3 times for 10 min each with PBS followed by secondary antibody diluted 1:100 in PBS. Probes included phalloidin (AlexaFluor 633) and Hoechst 33342 (Invitrogen) for nuclear/DNA labeling were used at a 1:1000 dilution in PBS. After incubation, cells were washed 3 times for 10 min each and mounted in ProLong Gold (Vector Laboratories, USA) overnight at 4° C.

### RNA Extraction and Quantitative PCR.

Media was aspirated from monolayers and both basolateral and apical sides were washed 1× with PBS Ambion PureLink RNA Mini Kit lysis buffer was added to each well per the manufacturer instructions. The buffer was used to gently dislodge the monolayer. The three lysis washes were collected in a 15 ml conical for RNA extraction. RNA was quantified with the Qubit® RNA HS Assay (Life Technologies). 1.0 μg of RNA was used in cDNA synthesis using Superscript IV Reverse Transcriptase (Life Technologies) following the manufacturer's standard protocol. Gene expression was evaluated using IDT PrimeTime qPCR Assays (IDT) following both the protocol and suggested cycling conditions for 10 μL reactions. qPCR was performed on the QuantStudio 12K Flex Real-Time PCR System (Applied Biosystems) and analyzed with the QuantStudio 12K Flex Software V1.2.2 (Applied Biosystems). Primers will be provided upon request.

### Statistical Analysis.

All the data are presented as mean +/− standard deviation (S.D.) or standard error of the mean (S.E.M.) and were analyzed using Prism software (GraphPad). Statistical significance of differences was assessed using Multiple Mann-Whitney tests or two-way ANOVA with Bonferroni's test for multiple comparisons and the significance cutoff was p<0.05.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

What is claimed is:

1. A method generating an intestinal organoid (IO) with immune cells (iIO) comprising transplanting an IO into an organism having immune cells for a period of time sufficient to form an IO having immune cells in one or more cell layers of the IO (iIO).

2. The method of claim 1, wherein the period of time is, is about, is at least, or is at least about, 6, 8, 10, 12, 14, 16, 18, or 20 weeks, or a range defined by any two of the preceding values, optionally, 6-20, 12-20 or 16-20 weeks.

3. The method of claim 1 or 2, wherein the period of time is, is about, is at least, or is at least about, 12 weeks or 16 weeks.

4. The method of any one of the preceding claims, wherein the iIO comprises CD4+ T cells and/or CD20+ B cells localized to lamina propria and epithelium.

5. The method of any one of the preceding claims, wherein the iIO comprises aggregates of T and B cells.

6. The method of any one of the preceding claims, wherein the iIO comprises CD4+ T cells and CD8+ T cells in a T-cell zone.

7. The method of any one of the preceding claims, wherein the iIO comprises plasma cells and neutrophils.

8. The method of any one of the preceding claims, wherein the iIO expresses gut-associated lymphoid tissue (GALT)-associated chemokines.

9. The method of claim 8, wherein the GALT-associated chemokines are CCL19, CCL21, and/or CXCL13.

10. The method of any one of the preceding claims, wherein the iIO comprises GALT-associated B cells.

11. The method of claim 10, wherein the B cells are aggregated in a lymphoid-like structure.

12. The method of any one of the preceding claims, wherein the iIO comprises CD45+ cells.

13. The method of claim 12, wherein the CD45+ cells are localized to a mucosal layer, lamina propria, and/or epithelium of the iIO.

14. The method of claim 12 or 13, wherein the CD45+ cells form cellular aggregates.

15. The method of any one of the preceding claims, wherein the iIO comprises CD3+ B cells and CD20+ T cells.

16. The method of any one of the preceding claims, wherein the iIO comprises CD4+ T cells, innate lymphoid cells, mucosal-associated invariant T (MAIT)-like cells, CD8+ T cells, lymphoid tissue inducer-like cells, B cells, natural killer cells, dendritic cells, intraepithelial lymphocytes, macrophages, natural killer T cells, and/or neutrophils.

17. The method of any one of the preceding claims, wherein the iIO comprises enterocytes, goblet cells, Paneth cells, and enteroendocrine cells.

18. The method of any one of the preceding claims, wherein the iIO comprises Villin+, Mucin2+, Lysozyme+, and/or Chromogranin A+ cells.

19. The method of any one of the preceding claims, wherein upon exposure of the lumen of the iIO to a lysate of Escherichia coli, microfold cells (M cells) of the iIO express glycoprotein 2 (GP2) at the cell surface.

20. The method of any one of the preceding claims, wherein upon exposure of the lumen of the iIO to a lysate of Escherichia coli, plasma cells of the iIO produce IgA antibodies.

21. The method of any one of the preceding claims, wherein upon exposure of the lumen of the iIO to a lysate of Escherichia coli, M cells and B cells of the iIO are co-localized.

22. The method of any one of the preceding claims, wherein the organism is not human.

23. The method of any one of the preceding claims, wherein the IO is transplanted into a kidney capsule of the organism.

24. The method of any one of the preceding claims, wherein the organism does not have Peyer's patches or lymphoid follicles in an intestine.

25. The method of any one of the preceding claims, wherein the organism is a mouse.

26. The method of claim 25, wherein the mouse is a lymphopenic mouse 27. The method of claim 26, wherein the mouse is a NOD/SCID/112rg−/− mouse with transgenic expression of human SCF, GM-CSF, and IL-3 (NSGS mouse).

28. The method of any one of the preceding claims, wherein prior to transplanting of the IO, the mouse is injected with cord blood cells, optionally wherein the cord blood cells are human cord blood cells.

29. The method of claim 28, wherein the mouse is treated with a pharmaceutical compound prior to the injection of cord blood cells to optimize engraftment of cord blood cells, optionally wherein the pharmaceutical compound depletes mouse bone marrow cells.

30. The method of any one of the preceding claims, wherein the IO is made by a method comprising:
a) exposing definitive endoderm (DE) cells to an FGF activator and a Wnt pathway activator for a period of time to differentiate into a hindgut spheroid, optionally a mid-hindgut spheroid;
b) embedding the hindgut spheroid into a basement membrane matrix; and
c) exposing the embedded hindgut spheroid to EGF for a period of time to differentiate into an IO, optionally wherein the IO is a human IO (HIO).

31. The method of any one of the preceding claims, wherein the method further comprises, prior to implanting the IO, making the IO by a method comprising:
a) exposing definitive endoderm (DE) cells to an FGF activator and a Wnt pathway activator for a period of time to differentiate into a hindgut spheroid, optionally a mid-hindgut spheroid;
b) embedding the hindgut spheroid into a basement membrane matrix; and
c) exposing the embedded hindgut spheroid to EGF for a period of time to differentiate into an IO, optionally wherein the IO is an HIO.

32. The method of claim 30 or 31, wherein the DE cells are derived from pluripotent stem cells (PSCs), optionally wherein the PSCs are embryonic stem cells (ESCs) or induced pluripotent stem cells (iPSCs), optionally wherein the PSCs are human PSCs.

33. The method of any one of claims 30-32, wherein the basement membrane matrix environment is Matrigel.

34. The method of any one of claims 30-33, wherein the FGF activator is FGF4, optionally wherein the concentration is, is about, is at least, or is at least about 50 ng/ml, 100 ng/ml, 150 ng/ml, 200 ng/ml, 250 ng/ml, 300 ng/ml, 350 ng/ml, 400 ng/ml, 450 ng/ml, 500 ng/ml, 550 ng/ml, 600 ng/ml, 650 ng/ml, 700 ng/ml, or 750 ng/ml, or a range defined by any two of the preceding values, optionally 50 -750 ng/ml, 50-100 ng/ml, or 50-500 ng/ml, or optionally at a concentration of 500 ng/ml.

35. The any one of claims 30-34, wherein the Wnt pathway activator is CHIRON 99021, optionally wherein the concentration is, is about, is at least, or is at least about 0.5 μM, 1 μM, 1.5 μM, 2 μM, 2.5 μM, 3 M, 3.5 μM, 4 μM, 4.5 μM, 5 μM, 5.5 μM, or 6 μM, or a range defined by any two of the preceding values, optionally 0.5-6 μM, 0.5-3 μM, 3-6 μM, 2-4 μM, or optionally at a concentration of 3 μM.

36. The method of any one of claims 30-35, wherein the concentration of EGF is, is about, is at least, or is at least about 25 ng/ml, 50 ng/ml, 75 ng/ml, 100 ng/ml, 125 ng/ml, 150 ng/ml, 175 ng/ml, or 200 ng/ml, or a range defined by any two of the preceding values, optionally 25-100 ng/ml, 50 -150 ng/ml, 100 ng/ml, or optionally is at a concentration of 100 ng/ml.

37. The method of any of the preceding claims, wherein the IO is matured in vitro for a period of time prior to transplantation, optionally wherein the period of time is, is about, is at least, or is at least about, 7, 10, 14, 16, 21, 25, or 28 days, or a range defined by any two of the preceding values, optionally 7-28, 14-28, or 21-28 days.

38. A method of making an iIO with activated immune cells comprising administering an immune stimulating material to a lumen of the iIO.

39. The method of claim 38, wherein the iIO is made by the method of any one of claims 1-37.

40. The method of claim 38 or 39, wherein the immune stimulating material is an allergen, and/or a microbial lysate, optionally wherein the microbial lysate comprises Escherichia coli.

41. The method of any one of claims 38-40, wherein the iIO with activated immune cells comprises GP2+ microfold (M) cells after a period of time post-administration of the microbial lysate.

42. The method of claim 41, wherein the period of time post-administration is, is about, is at least, or is at least about, 24, 36, 48, 60, or 72 hours.

43. The method of any one of claims 38-42, wherein mucus of the iIO with activated immune cells comprises secreted IgA antibodies.

44. The method of any one of claims 38-43, wherein M cells and B cells colocalize within the iIO with activated immune cells.

45. An iIO with activated immune cells made by the method of any one of claims 38-44.

46. The method of any one of the preceding claims, wherein the IO is a human IO (HIO).

47. The method of any one of the preceding claims, wherein the immune cells of the organism comprise human immune cells, and/or the organism has a humanized immune system.

48. The method of any one of the preceding claims, wherein the IO is an HIO, wherein the organism has human immune cells and/or a humanized immune system, and wherein the HIO comprises human immune cells and/or humanized immune cells in one or more layers of the HIO (hiHIO).

49. An iIO made by the method of any one of the preceding claims.

50. An IO comprising immune cells in one or more cell layers of the IO (iIO).

51. The iIO of claim 50, wherein the iIO comprises CD4+ T cells and/or CD20+ B cells localized to lamina propria and epithelium.

52. The iIO of any one of claims 50-51, wherein the iIO comprises aggregates of T and B cells.

53. The iIO of any one of claims 50-52, wherein the iIO comprises CD4+ T cells and CD8+ T cells in a T-cell zone.

54. The iIO of any one of claims 50-53, wherein the iIO comprises plasma cells and neutrophils.

55. The iIO of any one of claims 50-54, wherein the iIO expresses gut-associated lymphoid tissue (GALT)-associated chemokines.

56. The iIO of claim 55, wherein the GALT-associated chemokines are CCL19, CCL21, and/or CXCL13.

57. The iIO of any one of claims 50-56, wherein the iIO comprises GALT-associated B cells.

58. The iIO of claim 57, wherein the B cells are aggregated in a lymphoid-like structure.

59. The iIO of any one of claims 50-58, wherein the iIO comprises CD45+ cells.

60. The iIO of claim 59, wherein the CD45+ cells are localized to a mucosal layer, lamina propria, and/or epithelium of the iIO.

61. The iIO of claim 59 or 60, wherein the CD45+ cells form cellular aggregates.

62. The iIO of any one of claims 50-61, wherein the iIO comprises CD3+ B cells and CD20+ T cells.

63. The iIO of any one of claims 50-62, wherein the iIO comprises CD4+ T cells, innate lymphoid cells, mucosal-associated invariant T (MAIT)-like cells, CD8+ T cells, lymphoid tissue inducer-like cells, B cells, natural killer cells, dendritic cells, intraepithelial lymphocytes, macrophages, natural killer T cells, and/or neutrophils.

64. The iIO of any one of claims 50-63, wherein the iIO comprises enterocytes, goblet cells, Paneth cells, and enteroendocrine cells.

65. The iIO of any one of claims 50-64, wherein the iIO comprises Villint, Mucin2+, Lysozyme+, and/or Chromogranin A+ cells.

66. The iIO of any one of claims 50-65, wherein upon exposure of the lumen of the iIO to a lysate of Escherichia coli, microfold cells (M cells) of the iIO express GP2 at the cell surface.

67. The iIO of any one of claims 50-66, wherein upon exposure of the lumen of the iIO to a lysate of Escherichia coli, plasma cells of the iIO produce IgA antibodies.

68. The iIO of any one of claims 50-67, wherein upon exposure of the lumen of the iIO to a lysate of Escherichia coli, M cells and B cells of the iIO are co-localized.

69. The iIO of any one of claims 50-68, comprising activated immune cells.

70. The iIO of claim 69, wherein the iIO comprises GP2+ microfold (M) cells.

71. The iIO of any one of claims 69-70, wherein mucus of the iIO comprises secreted IgA antibodies.

72. The iIO of any one of claims 69-71, wherein M cells and B cells colocalize within the iIO.

73. The iIO of any one of claims 49-72, wherein the IO is an HIO.

74. The iIO of any one of claims 49-73, wherein the immune cells comprise human and/or humanized immune cells.

75. The iIO of any one of claims 49-74, wherein the IO is an HIO, and wherein the HIO comprises human immune cells and/or humanized immune cells in one or more layers of the HIO (hiHIO).

76. The method or iIO of any one of the preceding claims, wherein the iIO resembles at least one GI disease state.

77. The method or iIO of claim 76, wherein the GI disease state is an allergy and/or infectious disease.

78. The method or iIO of claim 76 or 77, wherein the GI disease state is induced by a genetic modification of one or more cell types of the iIO.

79. £ The method or iIO of any one of claims 76-78, wherein the GI disease state is induced by exposure to a compound, an allergen, and/or a pathogen.

80. The method or iIO of any one of the preceding claims, wherein the iIO is utilized to develop a mucosal vaccine.

81. A method of treatment comprising transplanting the iIO of any one of claims 49-75 into an organism, optionally wherein the organism is suffering from a GI disease state.

82. A method of screening a compound for activity comprising contacting the iIO of any one of claims 49-79 with the compound and measuring a response of the iIO to the compound.
